(19) United States
(12) Patent Application Publication
(10) Pub. No.: US 2023/0335258 A1
(43) Pub. Date: Oct. 19, 2023

(54) METHODS AND SYSTEMS FOR MULTI-OMIC INTERVENTIONS FOR MULTIPLE HEALTH CONDITIONS

(71) Applicant: Food Rx and AI, Inc., Mountain View, CA (US)

(72) Inventors: Ranjan Sinha, Los Altos Hills, CA (US); Shreyas V. Kumbhare, Maharashtra (IN); Inti Pedroso, Región del Maule (CL); Bharat Joshi, West Bengal (IN); Santosh K. Saravanan, Tamil Nadu (IN); Karthik M. Muthukumar, Tamil Nadu (IN); Daniel E. Almonacid, San Diego, CA (US)

(21) Appl. No.: 18/299,571

(22) Filed: Apr. 12, 2023

Related U.S. Application Data
(60) Provisional application No. 63/476,672, filed on Dec. 22, 2022.
(60) Provisional application No. 63/330,316, filed on Apr. 13, 2022.

(51) Int. Cl. G16H 20/70; G16B 40/20; G16H 50/20; G16B 20/20; G16H 20/60; G16H 50/30

(52) U.S. Cl. CPC G16H 20/70; G16B 20/20; G16B 40/20; G16H 20/60; G16H 50/20; G16H 50/30

(57) ABSTRACT

A platform providing methods and systems for prevention and/or treatment of a health condition (e.g., mental health condition and/or weight-related condition), where a method can include: simultaneously reducing severity symptoms of the health condition and comorbid conditions upon: receiving a set of samples from one or more subjects; receiving a biometric dataset from one or more subjects; receiving a lifestyle dataset from one or more subjects; returning a genomic single nucleotide polymorphism (SNP) profile and a baseline microbiome state upon processing the set of samples, the biometric dataset, and the lifestyle dataset with a set of transformation operations; generating personalized intervention plans for the one or more subjects upon processing the genomic SNP profile and the baseline microbiome state with a multi-omic model; and executing the personalized intervention plans for the one or more subjects.

### CROSS-REFERENCE TO RELATED APPLICATIONS

This application claims the benefit of U.S. Provisional Application No. 63/330,316 filed on 13-APR-2022 and U.S. Provisional Application No. 63/476,672 filed on 22-DEC-2022, which are each incorporated in its entirety herein by this reference.

### TECHNICAL FIELD

The disclosure generally relates to systems executing methods for sampling, processing, and identifying diagnostic and therapeutic signatures in patients, with actionable outcomes (e.g., in relation to prevention, diagnostic, and/or treatment of conditions), in the field of multi-omics.

### BACKGROUND

Mental health conditions are major contributors to morbidity and healthcare costs worldwide, and have significant detrimental impacts on society. Obesity and gut disorders are often comorbid with mental health conditions, and many factors contribute to key mechanistic roles in linking such disorders. Pharmacological and behavioral interventions are currently used to treat mental health conditions, but they have limited efficacy.

Poor mental health is a significant determinant of health-related quality of life, with important implications at individual and population levels. Pharmacological and behavioral interventions prevent and treat individuals at risk of or suffering from mental health disorders, but their efficacy has certain limits, and many subjects experiencing improvements will relapse. World events have worsened the mental health crisis and brought awareness to relationships between mental health and other chronic health conditions. There is a great need to develop cost-effective interventions that provide significant short and long-term therapeutic benefits to individuals suffering from mental health, especially those with multiple comorbid conditions. Digital therapeutics have gained increased attention as a strategy to provide care to large numbers of individuals, and emerging evidence suggests its effectiveness for several chronic diseases.

Relatedly, obesity is a major health problem due to its profound health deteriorating effects and high costs for health care systems. Obesity and related comorbidities are a public health priority with rising costs for private and public health systems. There exist lifestyle and pharmacological interventions available to prevent and reverse obesity, however, at the population level these have shown to be insufficient and a worldwide increase in obesity prevalence continues to be observed. There is a paucity of evidence regarding how to tailor the diet for an individual’s microbiome and what are changes expected to occur as a result of successful weight management.

Even more generally, healthcare systems domestically and internationally are impacted by large numbers of patients with preventable health conditions. Preventive healthcare approaches are thus being developed and participation is incentivized in order to proactively improve patient health states. Curative healthcare approaches are also useful in minimizing impact on healthcare systems, especially in the context of returning individuals to healthy states such that they no longer burden healthcare systems. While many preventative and curative healthcare approaches for prevention and treatment of chronic diseases center around behavioral and lifestyle changes, such approaches are often applied in a general and non-personalized manner. Furthermore, general and personalized approaches both suffer from patient compliance issues, limitations in contextual data streams used to drive successful outcomes, limitations in ability to provide just-in-time interventions, and other factors.

Genomic characterization of patients can be used for diagnostic purposes; however, such characterizations often provide a partial characterization of a patient’s state, in the interests of improving personalization of preventive and curative care options. Furthermore, methods for identification of diagnostic signatures from patient samples, and generation and application of therapeutic pathways (e.g., combinatorial therapies) tailored to specific patients have not been viable due to limitations in current technologies. There is a paucity of work covering the effectiveness of digital care based on genome SNP and microbiome markers to guide lifestyle and dietary modulations for improvement of mental health, weight, and/or other conditions with comorbidities, and on modeling outcomes based on a combination of these markers.

As such, there is a need in the field of multi-omics to characterize patient statuses and improve patient outcomes using personalized care approaches in a more informed manner.

### SUMMARY

The invention(s) include systems, methods, and/or architecture to deliver a digital therapeutics care program, with personalized interventions based upon weight loss models informed by demographic characteristics, engagement and behavioral, genetic, and baseline microbiome variables of subjects involved. Applications of the invention(s) identify and make use of genetic and gut microbiome factors that produce improvements in mental health conditions and weight conditions, for subjects who follow customized dietary and lifestyle interventions based upon outputs of models that process such genetic and gut microbiome factors as inputs.

The disclosure provides an embodiment of a method for prevention and treatment of a mental health condition, the method including: simultaneously reducing severity of a set of mental health condition symptoms and producing a reduction in body mass index (BMI) greater than a threshold, across a set of subjects upon: receiving a set of samples from the set of subjects; receiving a biometric dataset from the set of subjects; receiving a lifestyle dataset from the set of subjects; returning a genomic single nucleotide polymorphism (SNP) profile, a baseline microbiome state, and a set of signatures upon processing the set of samples, the biometric dataset, and the lifestyle dataset with a set of transformation operations; generating a personalized intervention plan for a subject of the set of subjects upon processing the genomic SNP profile, the baseline microbiome state, and the set of signatures with a multi-omic model; and executing the personalized intervention plan for the subject. In applications of use, the set of mental health conditions symptoms can include symptoms associated with one or more of: anxiety, depression, and sleep.

Mental health conditions can additionally or alternatively be associated with one or more of: obsessive compulsive disorders, mood disorders, bipolar disorder, Panic disorder, phobias (e.g., agoraphobia, etc.), substance use disorders, alcohol use disorders, attention deficit disorders, attention deficit hyperactivity disorders, stress related disorders, personality trait disorders, Tryptophan levels, Serotonin levels, Acetate levels, Butyrate levels, Propionate levels, and/or other conditions.

Comorbid conditions can additionally or alternatively be associated with one or more of: body mass index (BMI)-related disorders, obesity, functional gastrointestinal disorders (e.g., irritable bowel syndrome, inflammatory bowel disease, etc.), gastroesophageal reflux disease (GERD), non-alcoholic fatty liver disease, type I diabetes, type 2 diabetes, insulin resistance, and/or other conditions.

In a specific application, a group of 369 subjects participating in a personalized digital care program provided by the systems and methods described, were recruited. Samples and data processed from the subjects, with a multi-omic model (training of which is described in further detail below), generated associations between a number of genetic factors (e.g., 23 or more genetic factors), an abundance of microbiome genera and other taxa (e.g., 178 gut microbiome genera), and a set of gut-brain pathways related to neuroactive metabolites produced by gut microbes (e.g., 42 gut-brain pathways or more), in relation to the presence and/or absence of anxiety, depression, or sleep problems at baseline, during program involvement, and post-program involvement. Identified genetic factors, microbiome factors, and gut-brain pathways were used to customize interventions, which resulted in significant improvements in symptoms of anxiety, depression, and sleep/insomnia, along with healthy losses in body weight (e.g., 2% body weight reductions, reductions in BMI by 2 or more BMI units).

In relation to the specific application, the mean BMI and age of the study cohort were 34.6 and 48.7, respectively, and there was an overrepresentation of individuals with functional gastrointestinal disorders 84%). On average, the individuals lost 5.4% of body weight at the time of follow-up (mean of 88 days), and more than 95% reported improvement in at least one outcome. Significant correlations between genetic factors with anxiety and depression at baseline, gut microbial functions with sleep problems at baseline, and genetic factors and gut microbial taxa and functions with anxiety, depression, and insomnia improvement were identified and used to generate aspects of customized interventions for subjects. Among the gut microbial functions identified, the abundance of butyrate synthesis genes was associated with improvement in depression symptoms, the abundance of kynurenine synthesis genes was associated with improvement in anxiety symptoms, and the abundance of genes able to synthesize and degrade neuroactive hormones, such as nitric oxide, were associated with improvement in depression and insomnia symptoms. Among the genetic factors identified, anxiety or depression at baseline was associated with genetic scores for alcohol use disorder and major depressive disorder, and improvement in anxiety and depression symptoms was associated with obstructive sleep apnea presence. Furthermore, Improvement of insomnia symptoms was associated with both a type 1 and a type 2 diabetes genetic factor. The relative ability of demographic, genetic, and microbiome factors to explain baseline states of subjects, and to be used as inputs for models with architecture for guiding improvements in mental health was also identified.

In examples, the invention(s) were able to achieve simultaneous reductions in severity of anxiety symptoms by at least 59%, depression symptoms by at least 51%, and insomnia symptoms by at least 66% across the population of subjects, along with weight reductions described. Variations of the specific application can be applied to other numbers of groups of subjects.

As such, the digital therapeutics care program significantly decreased body weight in a healthy manner, and concomitantly decreased mental health symptom intensity (e.g., based upon self-reported data from subjects, based upon non-self reported data from caretakers of subjects, based upon detection of symptom severity from biometric monitoring devices). Results of the invention(s) provide evidence that genetic and gut microbiome factors help explain inter-individual differences in mental health improvement after dietary and lifestyle interventions for weight loss. Thus, individual genetic and gut microbiome factors provide a basis for designing and further personalizing dietary interventions to improve mental health.

The invention(s) further identified gut network enterotypes and their relationships with mental health improvement. In a specific example, gut network enterotypes and relationships were identified using an ecological network analysis model that implemented a top-down approach to process complex microbiome interactions, where training of machine learning models identified the ecological groups correlated with mental health conditions and BMI, and the key drivers of those sub-communities. Trained models were structured to return valuable biomarkers, including new targets for synbiotics to modify community patterns, with reductions in data complexity with graph analyses. Network Module communities associated with mental health and BMI conditions, along with direction of an effect upon such conditions, are further described in more detail below, where each community has a set of microbiome features associated with it and also a subset of microbiome features that are representative of characteristics of the community, such that they can be considered the most relevant. Prebiotic or other food components known to impact those network module communities and microbiome features (e.g., by impacting key taxa drivers) were used to generate personalized intervention plan elements to improve outcomes.

In an embodiment, the inventions include: a method for prevention and treatment of a mental health condition, the method comprising: simultaneously reducing severity of a set of mental health condition symptoms by at least 50% and producing a reduction in body mass index (BMI) greater than 2 BMI units, across a set of subjects upon: receiving a set of samples from the set of subjects; receiving a biometric dataset from the set of subjects; receiving a lifestyle dataset from the set of subjects; returning a genomic single nucleotide polymorphism (SNP) profile, a baseline microbiome state, and a set of signatures upon processing the set of samples, the biometric dataset, and the lifestyle dataset with a set of transformation operations; generating a personalized intervention plan for a subject of the set of subjects upon processing the genomic SNP profile, the baseline microbiome state, and the set of signatures with a multi-omic model; and executing the personalized intervention plan for the subject.

In an embodiment, the inventions include: a method for prevention and treatment of a mental health condition, the method comprising: simultaneously reducing severity of a set of mental health condition symptoms, comprising symptoms of anxiety, depression, and sleep, by at least 50% across a population of subjects upon: receiving a set of samples from the population of subjects, the set of samples comprising saliva samples and gut samples; receiving a biometric dataset from the population of subjects, the biometric dataset comprising BMI values; receiving a lifestyle dataset from the population of subjects; returning a genomic single nucleotide polymorphism (SNP) profile, a microbiome state, and a set of signatures upon processing the set of samples, the biometric dataset, and the lifestyle dataset with a set of transformation operations; generating a personalized intervention plan for a subject of the set of subjects upon processing the genomic SNP profile, the microbiome state, and the set of signatures with a multi-omic model; and executing the personalized intervention plan for the subject.

In an embodiment, the inventions include: a method for prevention and treatment of a health condition, the method comprising: producing a reduction in body mass index (BMI) greater than an average 2 BMI units across a population of subjects upon: receiving a set of samples from the population of subjects, the set of samples comprising saliva samples and gut samples; receiving a biometric dataset from the population of subjects, the biometric dataset comprising BMI values and blood glucose values; receiving a lifestyle dataset from the population of subjects; returning a genomic single nucleotide polymorphism (SNP) profile, a microbiome state, and a set of signatures upon processing the set of samples, the biometric dataset, and the lifestyle dataset with a set of transformation operations; generating a personalized intervention plan for a subject of the set of subjects upon processing the genomic SNP profile, the microbiome state, and the set of signatures with a multi-omic model; and executing the personalized intervention plan for the subject.

The invention(s) generally provide and execute personalized digital care interventions that uses its multi-omics platform to provide dietary and lifestyle recommendations that are personalized using genetic and microbiome information. The invention(s) covered herein further address gut microbiome taxa or functions and genetic markers in affecting body weight loss and relatedly, effects on mental health and weight-related health issues.

The invention(s) generally provide and execute personalized digital care interventions in order to increase population access to care, reduce costs, and improve engagement with intervention programs. Provided herein are methods, systems, and compositions for providing dietary and lifestyle interventions to achieve weight loss that are effective in reducing weight and improving diverse health outcomes, by prioritizing and personalizing food ingredients to match subjects’ genetic profiles and nurture their respective gut microbiomes.

Additional aspects and advantages of the present disclosure will become readily apparent to those skilled in this art from the following detailed description, wherein only illustrative embodiments of the present disclosure are shown and described. The present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference in their entireties for all purposes and to the same extent as if each individual publication, patent, or patent application was specifically and individually indicated to be incorporated by reference. Furthermore, where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

### BRIEF DESCRIPTION OF THE FIGURES

FIG. 1A depicts an embodiment of a workflow of a method for obtaining diagnostic and therapeutic signatures for a subject based on multi-omic models and executing a personalized intervention plan.

FIG. 1B depicts an embodiment of training of the multi-omic models used in a workflow of a method for obtaining diagnostic and therapeutic signatures of a subject.

FIG. 1C depicts a variation of a workflow of a method for preventing and/or treating mental health and/or BMI-related conditions based on multi-omic models, and executing a personalized intervention plan.

FIG. 2 depicts a graphical abstract of an embodiment of the invention(s), for providing personalized care for various health conditions.

FIG. 3 depicts an exemplary framework for evaluating symptom severity and changes in subjects suffering from mental health conditions.

FIG. 4 depicts exemplary changes in subjects suffering from depression, anxiety, and or sleep-related health conditions, for subjects who also surpassed threshold reductions in BMI/weight when participating in personalized intervention plans.

FIG. 5 depicts performances of various models for predictions and generation of personalized intervention plan features, where models incorporate combinations of genetic and microbiome factors.

FIG. 6 depicts exemplary aspects of factors contributing to generation of personalized intervention plans for subjects suffering from conditions described.

FIG. 7 depicts an embodiment of a framework for providing personalized intervention plans to subjects, with involvement of large language models.

FIG. 8 depicts exemplary data covering factors contributing to, associated with, and resulting in improvements in BMI/weight-related issues.

FIG. 9A depicts an embodiment of a system implementing multi-omic models to generate personalized intervention plans for subjects suffering from conditions described.

FIG. 9B depicts an embodiment of computing and control system elements for generating to generate personalized intervention plans for subjects suffering from conditions described.

### DESCRIPTION OF THE EMBODIMENTS

While various embodiments of the invention have been shown and described herein, it will be obvious to those skilled in the art that such embodiments are provided by way of example only. Numerous variations, changes, and substitutions can occur to those skilled in the art without departing from the invention. It should be understood that various alternatives to the embodiments of the invention described herein can be employed.

### 1. Benefit(s)

The invention(s) provide systems and methods for generating multi-omic (i.e., genomic, microbiome analysis-based, behavior-based, lifestyle-based, clinical marker-based, etc.) diagnostics and therapeutic pathways for precision care (e.g., with respect to prevention, diagnosis, and/or treatment of conditions) in response to various indications. In examples, characterizations can be used to provide actionable insights, with example executed actions including recommendations of inexpensive dietary and lifestyle therapeutic interventions. Additionally or alternatively, characterizations and data processing from subjects can further reveal more complex diagnostic pathways and identify potential therapeutic pathways.

In particular, genetic and non-genetic factors contribute to the etiology of mental health disorders. Aspects of the inventions described include sample and data processing with trained models structured to link mental health condition symptoms with factors derived from genetics, microbiome states, behavior, and other factors, in order to generated customized interventions for improving states of subjects.

In specific examples, the invention(s) can be used to provide improved outcomes for subjects with one or more of: mental health conditions (e.g., anxiety, depression, autism, bipolar disorder, memory loss, brain fog, cognitive functions, etc.); sleep issues (e.g., sleep apnea, disturbed sleep, fatigue, etc.); weight-related issues (e.g., obesity, eating disorders, body dysmorphia, etc.), and other conditions.

The invention(s) can additionally or alternatively be used to provide improved outcomes for subjects with one or more of: cardiovasular and cardiometabolic disorders (e.g., hypertension, high LDL cholesterol, low HDL cholesterol, high triglycerides, etc.); insulin-related health conditions (e.g., type 2 diabetes, other forms of diabetes, prediabetes, polycystic ovary syndrome (PCOS), etc.);obesity or another overweight condition; chronic pain (e.g., joint pain including persistent joint aches and joint swellings, osteoarthritis, gout, rheumatoid arthritis, headaches, etc.); digestive health issues (e.g., irritable bowel syndrome (IBS), inflammatory bowel disease (IBD), acid reflux, gastroesophageal reflux disease (GERD), functional gastrointestinal disorders (FGIDs) such as functional constipation, functional diarrhea, bloating, etc.); skin health conditions (e.g., rashes/dryness, itching, hair loss, acne, rosacea, eczema, atopic dermatitis, psoriasis, alopecia, etc.); renal disorders (e.g., kidney stones, renal failure, etc.); non-alcoholic fatty liver disease (NAFLD); hormonal health conditions (e.g., hyperthyroidism, hypothyroidism, menopause, altered testosterone levels, etc.); and/or other conditions. Such conditions can be comorbid with mental health conditions, sleep conditions, and weight-related conditions described, and the inventions can simultaneously improve states of multiple conditions in an unprecedented manner, with respect to efficacy.

In more detail, embodiments, variations, and examples of a digital therapeutics program, informed by outputs of models structured to process genomic loci of interest (SNPs), gut microbiome information, and interactions with participant diet, demographics, and lifestyle, were used to classify and achieve significant reductions in symptomatology of described conditions (e.g., mental health conditions, sleep conditions, weight-related conditions) in comparison to non-treatment groups, with maintained results over time. The methods and models disclosed herein can readily be implemented to study other comorbidities where genetics and gut microbiome also play a central role in disease etiology.

Embodiments, variations, and examples of methods and models for identifying weight loss patterns and genomic and microbiome profiles of individuals to identify the effects of respective interventions on their gut microbiome between their baseline and follow-up samples, and health outcomes in relation to weight-related issues. In relation to performance achievements that were previously unattainable without the invention(s), 80% of individuals lost weight during exemplary studies (several of which are described). Model-returned analyses of their gut microbiome identified genera, functional pathways and microbial communities associated with BMI changes and dietary and lifestyle intervention. The microbial genera and functional pathways identified and implemented with trained models for generating personalized interventions for reducing BMI included: Akkermansia, Christensenella, Oscillospiraceae, Alistipes, and Sutterella, short-chain fatty acid (SCFA) production and degradation of simple sugars like arabinose, sucrose, and melibiose. Network analysis identified two microbiome communities associated with BMI, one of which also significantly responded to the intervention, which includes multiple known associations with BMI and obesity.

The disclosure covers models and training of models for identifying previously-unidentified biomarkers for improving health conditions described, that were undiscovered prior to creation of the invention(s).

The disclosure covers dietary interventions, including modulation of the types and amounts of food that are consumed, for affecting the composition and function of the gut microbiome, as well as insights into how these differences evolve over time and whether they play a role in the development or reversal of obesity.

The disclosure provides methods, systems, and compositions, where returned outputs enable identification of the effects of a personalized dietary intervention on the gut microbiome composition over time and its relationship to body mass index (BMI). To understand the effect of our dietary and lifestyle intervention designed to promote weight loss on the gut microbiome, methods include performance of microbiome testing at a first time point (e.g., beginning of a program at intake), and then repeating testing at one or more subsequent time points (e.g., approximately six months into the intervention, additional time points). Returned analyses focused on changes over time in the relative abundances of microbial taxa and their encoded metabolic pathways, alpha and beta diversity, and the structure of microbial co-abundance networks in response to dietary interventions.

The invention(s) also confer the benefit of providing improved methods and systems for generation of diagnostic and therapeutic signatures from multi-omic data. Diagnostic signatures (described in more detail below) are returned by data transformation architecture, and in embodiments, can be indicative of decreased/increased likelihood of developing a health condition. Therapeutic signatures (described in more detail below) are returned by data transformation architecture and in embodiments, can be indicative of decreased/increased likelihood of improvement or remission of a health condition.

The invention(s) also confer the benefit of applying model outputs generated upon processing such signatures to develop new pharmaceutical, nutraceutical, skinceutical, and/or other compositions, including but not limited to chemical compositions (e.g., small molecule modulators) and biological compositions (e.g., pre-/pro-/syn-/post-biotics). Outputs of the inventions described can provide improved and personalized recommendations in relation pharmagogenomics for specific subjects.

Additionally, in embodiments, the invention(s) described implement rapid processing of samples and data generated from sample processing, in order to detect presence of biomarkers from patients, and extract insights for diagnostic and therapeutic applications, in a manner that cannot be practically performed in the human mind.

Additionally, the inventions apply outputs of the analyses to effect one or more actions (e.g., therapeutic pathways) for improvement of patient health statuses and outcomes, with specific treatments.

Additionally, the invention(s) involve collection of samples from patients and processing of other contextual data (e.g., behavior data, lifestyle-associated data), processing of samples to extract signatures, application of one or more transformations to the signatures to generate modified digital objects, create improved training data sets for machine learning/classification algorithms, and iteratively train the machine learning/classification algorithms with feedback, such that patient statuses can be returned upon processing subsequent samples hitherto unseen by the algorithm. Additionally, in embodiments, the invention(s) described implement rapid processing of samples and data generated from sample processing, in order to extract insights related to contributing factors to subject health, and rapid generation of personalized care program components, in a manner that cannot be practically performed by the human mind.

Additionally, the invention(s) include a platform for coordinating sampling from patients, processing of samples, generating multi-omic signatures from sample processing, returning insights upon processing multi-omic signatures with trained models, and applying therapeutic pathways for patients in a customized manner, by way of an application environment (e.g., mobile application environment, web application environment, etc.), coaching, and/or connected devices.

Additionally or alternatively, the invention(s) can confer any other suitable benefit.

### 1.1 Definitions

Multi-omics Intervention as a diagnostic approach combines one or more of: genome factors, microbiome factors, lifestyle factors, clinical markers, and other factors. The multi-omics approach described herein allows identification of diagnostic and therapeutic signatures in patients and/or other subjects.

The term “microbiome”, as used herein, refers to the ecological community of commensal, symbiotic, or pathogenic microorganisms in a sample.

The terms microbiome, microbiome information, microbiome data, microbiome population, microbiome panel and similar terms are used in the broadest possible sense, unless expressly stated otherwise, and would include: a census of currently present microorganisms, both living and non-living, which may have been present previously; a census of components of the microbiome other than bacteria and archaea (e.g., viruses, microbial eukaryotes, etc.); population studies and characterizations of microorganisms, genetic material, and biologic material; a census of any detectable biological material; and information that is derived or ascertained from genetic material, biomolecular makeup, fragments of genetic material, DNA, RNA, protein, carbohydrate, metabolite profile, fragment of biological materials and combinations and variations of these.

Gut flora (e.g. gut microbiota, gastrointestinal microbiota, etc.) is the complex community of microorganisms that live in the digestive tracts of humans and other animals, including insects. Gut metagenome is the aggregate of all the genomes of gut microbiota.

As used herein, terms real-time microbiome data or information includes microbiome information that is collected or obtained at a particular stage of the preventive or curative care of an individual.

As used herein, the terms derived microbiome information and derived microbiome data are to be given their broadest possible meaning, unless specified otherwise, and includes any real-time, microbiome information that has been computationally linked or used to create a relationship.

As used herein, the terms predictive microbiome information and predictive microbiome data are to be given their broadest possible meaning, unless specified otherwise, and includes information that is based upon combinations and computational links or processing of historic, predictive, real-time, and derived microbiome information, data, and combinations, variations and derivatives of these, which information predicts, forecasts, directs, or anticipates a future occurrence, event, state, or condition, or allows interpretation of a current or past occurrence.

Real time, derived, and predicted data can be collected and stored, and thus, become historic data for ongoing or future decision-making for a process, setting, or application.

The term “genome” as used herein, refers to the entirety of an organism’s hereditary information that is encoded in its primary DNA sequence. The genome includes both the genes and the non-coding sequences. For example, the genome may represent a microbial genome or a mammalian genome.

“Nucleic acid,” “oligonucleotide,” and “polynucleotide” refer to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. The term nucleic acid is used interchangeably with genetic material, cDNA, and mRNA encoded by a gene.

Reference to “DNA region” should be understood as a reference to a specific section of DNA. These DNA regions are specified either by reference to a gene name, a set of chromosomal coordinates or Reference single nucleotide polymorphisms (SNPs). Both the chromosomal coordinates and the gene or genomic region would be well known to, and understood by, the person of skill in the art. In general, a gene or genomic region can be routinely identified by reference to its name, via which both its sequences and chromosomal location can be routinely obtained, or by reference to its chromosomal coordinates, via which both the gene or genomic region name and its sequence can also be routinely obtained.

Reference to each of the genes/DNA regions detailed above should be understood as a reference to all forms of these molecules and to fragments or variants thereof. As would be appreciated by the person of skill in the art, some genes are known to exhibit allelic variation. Allelic variations encompass single nucleotide polymorphisms, insertions and deletions of varying size and simple sequence repeats, such as dinucleotide and trinucleotide repeats. Variants include nucleic acid sequences from the same region sharing at least 90%, 95%, 98%, 99% sequence identity i.e. having one or more deletions, additions, substitutions, inverted sequences etc. relative to the DNA regions described herein. Accordingly, the present invention should be understood to extend to such variants which, in terms of the present applications, achieve the same outcome despite the fact that minor genetic variations between the actual nucleic acid sequences may exist between different individuals of the same species (e.g., between different human subjects) or across different species (e.g., between different bacterial strains). The present invention should therefore be understood to extend to all forms of DNA which arise from any other mutation, polymorphic or allelic variation.

The term “sequencing” as used herein refers to sequencing methods for determining the precise order of the nucleotide bases-adenine, guanine, cytosine, and thymine-in a nucleic acid molecule (e.g., a DNA or RNA nucleic acid molecule).It includes any method or technology that is used to determine the order of the four bases-adenine, guanine, cytosine, and thymine-in a strand of DNA. The advent of rapid DNA sequencing methods has greatly accelerated biological and medical research and discovery.

The term “barcode” as used herein, refers to any unique, non-naturally occurring, nucleic acid sequence that may be used to identify the originating source of a nucleic acid fragment.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

### 2. Methods for Multi-Omic Interventions For Various Health Conditions

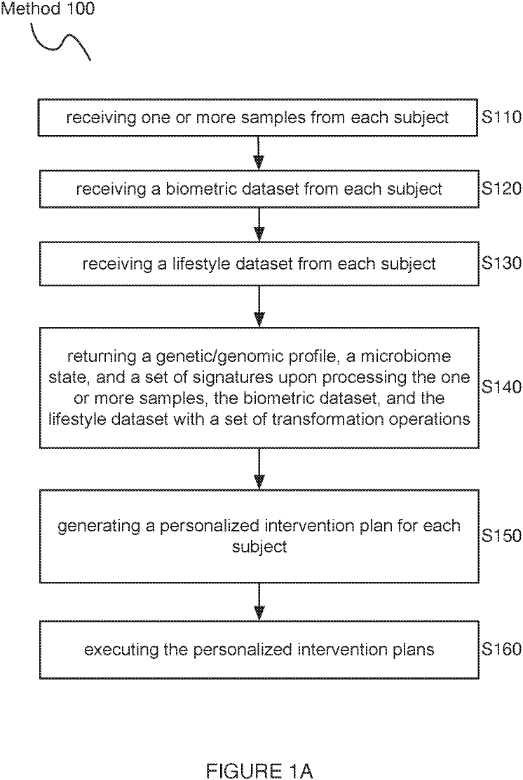

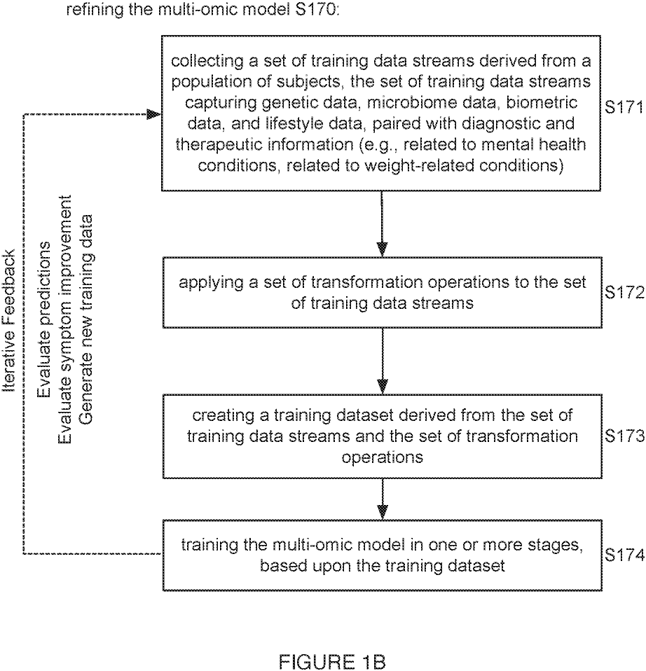

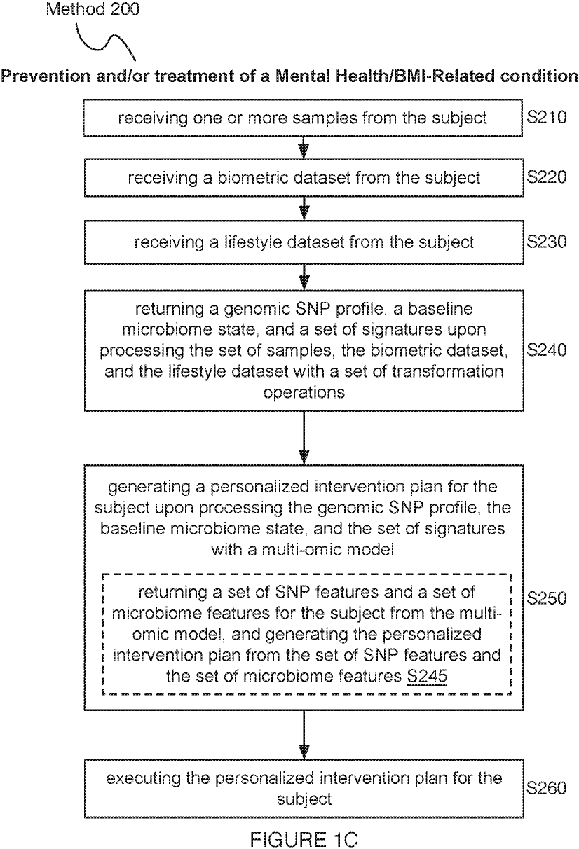

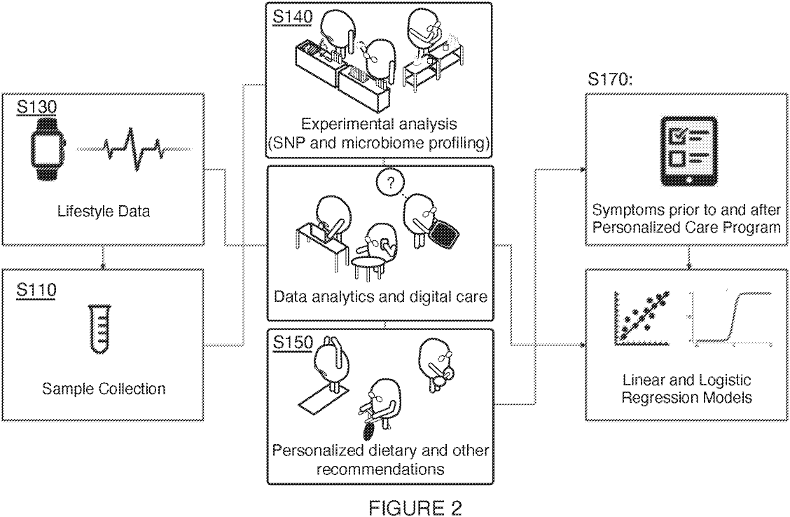

As shown in FIGS. 1A and 2, an embodiment of a method 100 includes (for each of a set of subjects): receiving a set of samples (e.g., one or more) from the subject S110; receiving a biometric dataset from the subject S120; receiving a lifestyle dataset from the subject S130; returning a genomic profile, a baseline microbiome state, and a set of signatures (e.g., including diagnostic signatures and/or therapeutic signatures) upon processing the set of samples, the biometric dataset, and the lifestyle dataset with a set of transformation operations S140; generating a personalized intervention plan for the subject upon processing the genomic profile, the baseline state, and the set of signatures with a multi-omic model S150; and executing the personalized intervention plan for the subject S160.

In some variations, as shown in FIG. 1B, the method 100 can further include refining the multi-omic model S170, wherein refining the multi-omic model includes: collecting a set of training data streams derived from a population of subjects, the set of training data streams capturing genetic data, microbiome data, biometric data, and lifestyle data, paired with diagnostic and therapeutic information, from the population of subjects S171, applying a set of transformation operations to the set of training data streams S172, creating a training dataset derived from the set of training data streams and the set of transformation operations S173, and training the multi-omic model in one or more stages (e.g., iteratively), based upon the training dataset S174 and/or additional training data.

In some variations, as shown in FIG. 1C, a method 200 for prevention and/or treatment of a mental health condition can include: simultaneously reducing severity of a set of mental health condition symptoms (e.g., by at least 50%) and/or producing a reduction in body mass index (BMI) greater than a threshold (e.g., at least 1 BMI unit, at least 2 BMI units), across a set of subjects including a subject, upon: receiving a set of samples from the subject S210; receiving a biometric dataset from the subject S220; receiving a lifestyle dataset from the subject S230; returning a genomic profile, a baseline microbiome state, and a set of signatures upon processing the set of samples, the biometric dataset, and the lifestyle dataset with a set of transformation operations S240; generating a personalized intervention plan for the subject upon processing the genomic profile, the baseline microbiome state, and the set of signatures with a multi-omic model S250; and executing the personalized intervention plan for the subject S260. In variations, the method 200 can include returning a set of genomic features and a set of microbiome features for the subject from the multi-omic model, and generating the personalized intervention plan from the set of genomic features and the set of microbiome features S245.

Embodiments, variations, and examples of the method 100 function to generate multi-omic (i.e., genomic, microbiome analysis-based, behavior-based, lifestyle-based, clinical marker-based, etc.) diagnostics and therapeutic pathways for precision care (e.g., prevention, diagnosis, and/or treatment) in response to various indications. The method 100 can generate multi-omic signatures (e.g., from individual biomarkers or combinations of genetic biomarkers, microbiome biomarkers, lifestyle biomarkers, etc.), in order to generate diagnostics and therapeutic pathways for subjects in a personalized manner. As such, the method 100 can improve patient outcomes with respect to mental health conditions, comorbid conditions, and/or various health states and indications, thereby addressing impacts on healthcare systems with personalized preventive healthcare approaches in an improved manner.

Variations of the methods described can be implemented for different subject types, different demographics, different health states, different lifestyles, and/or other factors (e.g., social determinants of health, work determinants of health, familial determinants of health, etc.), with specific applications for improving health statuses in relation to various conditions and indications described in more detail below.

Furthermore, in downstream applications, refinement of models, system architecture, and sample processing techniques can be used to guide testing of, recommendation of, and/or implementation of (e.g., using automated or manual systems/devices) therapeutic interventions in order to improve desired outcomes (e.g., in relation to patient adherence to regimens, in relation to patient engagement, in relation to remission and reduction of comorbidities, etc.). As such, the method(s) can provide steps for monitoring, controlling, and analyzing patient data and lifestyles, with practical applications in personalized preventive care for extremely large contributors to impacts on society and healthcare systems.

In specific use cases described below, the method 200 was applied to a set of 369 subjects, who lost greater than 2% body weight (or values of BMI units described) from a baseline state prior to start of respective personalized care programs and significantly reduced severity of mental health issues in comparison to baseline measured states, based upon identification and use of: (a) genetic scores derived from genetic factors identified from subjects with anxiety, depression and/or sleep in initial baseline measurements (e.g., prior to receiving personalized interventions) and gut microbial functions from microbiome factors identified from subjects with anxiety, depression and/or sleep in initial baseline measurements; (b) genetic scores derived from genetic factors identified from subjects with anxiety, depression and/or sleep, and microbiome taxonomic and functional features and functions identified from subjects with anxiety, depression and/or sleep, upon improvement of symptoms (e.g., upon, during, or after receiving personalized interventions). Personalized interventions and characterizations for each of the set of subjects were generated using model architecture described below, where training of such models and model architecture aspects improved the accuracy of predicting improvements in mental health symptomatology, based upon refinement and iterative training.

Additionally, specific examples of the method 200 were applied to a set of subjects for which level of body weight was a concern, and where subjects had comorbid health conditions (e.g., mental health conditions, other health conditions described), and use of specific examples of the method resulted in 80% of subjects losing weight greater than a threshold level (e.g., a reduction in BMI from a first time point to a second time point associated with receiving a personalized intervention was on average 2.57 BMI units). The specific use cases used an ecological network approach in implemented model architecture, to identify microbiome features (e.g., several taxonomic groups, including bacterial genera such as Christensenella, members of the Oscillospiraceae family and other taxonomic groups described below) which were used to generate intervention plan aspects that resulted in reduction in BMI across subjects. Additional microbiome features include gut microbial functions, such as mucin degradation and anti-inflammatory Short-Chain Fatty Acids (SCFAs) synthesis (and other functions described below) in relation to reductions in BMI, as well as simple sugar metabolism functions (e.g., associated with degradation) in relation to increases in BMI and higher energy extraction. Additional features include pathways, such as propionate synthesis pathways negatively associated with BMI, where propionate is a type of SCFA and increased gut levels were shown in personalized interventions to reduce inflammation, improve insulin sensitivity, and regulate appetite and body weight maintenance by promoting the secretion of Peptide YY (PYY) and Glucagon-like peptide-1 (GLP-1). Additional pathways identified included histamine synthesis pathways (e.g., declining histamine synthesis) and nitric oxide degradation pathways (e.g., increasing nitric oxide synthesis) in response to personalized interventions, with additional directional shifts in various microbiome pathways associated with metabolic, gut health, and mental health in association with reductions in BMI.

The method(s) described can be implemented by systems and platforms described in Section 3 below.

### 2.1 Methods - Sample, Biometrics, and Lifestyle Data Extraction

### 2.1.1 Sampling

Block S110 recites: receiving one or more samples from the subject, which functions to enable generation of a baseline state and one or more signatures from which models for returning diagnostics and personalized therapeutic pathways can be generated in subsequent portions of the method 100. In Block S110, samples can be retrieved from the subject (or each of a population of subjects) in a non-invasive manner. In variations, non-invasive manners of retrieving the one or more samples can implement one or more of: an absorbent material (e.g., a swab, a sponge, etc.), a non-absorbent material (e.g., a slide, etc.), a container (e.g., vial, tube, bag, etc.) configured to receive a sample from a region of a subject’s body, and any other suitable sample-receiver. In variations, samples can be collected from one or more of a subject’s mouth (e.g., a saliva sample), genitals, nose, skin, gut (e.g., through a stool sample, through a fecal sample, etc.) in a non-invasive manner. However, one or more samples can additionally or alternatively be received in a semi-invasive manner (e.g., glucose monitor, dry blood spots, etc.) or an invasive manner (e.g., blood sample, biopsy sample, etc.).

In above variations and examples, samples can be taken from the bodies of subjects without facilitation by a healthcare entity or another entity. In one example, a sampling kit can be provided to a subject, where the sampling kit facilitates self-sampling of genetic and/or microbiome samples from the subject. The sampling kit can further provide devices configured to facilitate reception of the biometric dataset and the lifestyle dataset from the subject in subsequent method blocks. The sampling kit can further include instructions for sample provision and setup of a user account within a platform for providing insights and care, elements configured to associate the sample(s) with the subject (e.g., barcode identifiers, tags, etc.), and a receptacle that allows the sample(s) from the subject to be delivered to a sample processing operation (e.g., by a mail delivery system). In another example, wherein samples are extracted from the user with the help of another entity, one or more samples can be collected in a clinical or research setting from a subject.

Samples can be collected once (e.g., at a single time point), or at a number of time points (e.g., at random points, at regular points, in relation to triggering events, with other frequency, etc.).

In expanded versions of Block S110 (e.g., with respect to generation of training and test datasets for model refinement in other portions of the method 100), samples can be retrieved from a wide variety of subjects with various conditions, demographics, and/or lifestyles, and can involve samples from human subjects and/or non-human subjects. In relation to human subjects, Block S110 can include receiving samples from a wide variety of human subjects, collectively including subjects of one or more categories such as: demographics (e.g., genders, ages, marital statuses, ethnicities, nationalities, socioeconomic statuses, sexual orientations, etc.), health conditions (e.g., health and disease states), living arrangements (e.g., living alone, living with pets, living with a significant other, living with children, etc.), environmental exposures (e.g. exposure to heavy metals, pesticides, xenobiotics etc.), dietary habits (e.g., omnivorous, vegetarian, vegan, sugar consumption, acid consumption, etc.), lifestyle tendencies (e.g., levels of physical activity, drug use, alcohol use, etc.), levels of activity, analyte states (e.g., cholesterol levels, lipid levels, hormone levels, etc.), weight, height, body mass index, genotypic factors, and any other suitable category.

Additionally or alternatively, receiving samples can include receiving samples from a targeted group of similar subjects to the subject, based on family relationships (e.g., twin relationships, triplet relationships, quadruplet relationships, etc., parental relationships, offspring relationships, etc.), subjects cohabiting with the subject, or subjects associated with the subject by other relevant linkages.

However, receiving the samples in Block S110 can additionally or alternatively be performed in any other suitable manner. Furthermore, some variations of the first method 100 can omit Block S110, or acquire sample data in another suitable manner.

### 2.1.2 Biometrics and Demographics

Block S120 recites: receiving a biometric dataset from the subject, which further functions to enable generation of a baseline state and one or more signatures from which models for returning diagnostics and personalized therapeutic pathways can be generated in subsequent portions of the method 100.

In variations, the biometric dataset can include data derived from one or more of: body weight (e.g., receiving bodyweight values of the subjects generated from a digital weighing scale), body fat percent, muscle mass, body water, height or other length measurements (e.g., via a ruler or measuring tape), other body mass index (BMI)-associated parameters, gastrointestinally-derived signals, Bristol stool scores, stool frequency, abdominal pain intensity, blood chemical and biochemical information, inflammatory markers, menstruation factors, hormonal factors, fasting blood sugar, high density lipids, low density lipids, fecal calprotectin, blood interleukins, c-reactive protein, blood cell counts, sleep-associated signals, electrophysiology signals (e.g., electroencephalogram signals, electromyography signals, galvanic skin response signals, electrocardiogram signals, etc.), heart rate, body temperature, cardiovascular parameters, continuous glucose monitoring (glycemic response), respiration parameters (e.g., respiration rate, depth/shallowness of breath, etc.), blood oxygenation signals, motion parameters, and any other suitable physiologically relevant parameter of the subject.

In examples, the biometric dataset can include data derived from one or more of: blood chemical and biochemical profiles, medication use, fasting blood sugar, glycemic response, high density lipid values, and low density lipid values.

As such, receiving the biometric dataset can include receiving a bodyweight value from at least one of the population of subjects, generated from a digital weighing scale, wherein the biometric dataset comprises a BMI value, and receiving a blood glucose value from at least one of the population of subjects, generated from a continuous glucose monitor, wherein the biometric dataset comprises a blood glucose value.

Additionally or alternatively, the biometric dataset can include or otherwise detect cyclic biometrics or biometrics that occur with some pattern (e.g., derived from user inputs, biometric monitoring devices worn by the user, or determined by an algorithm that tracks the user’s physical, emotional, or neurological states).

As described above, the sampling kit that facilitates self-sampling of genetic and/or microbiome samples from the subject can further include devices configured to facilitate reception of the biometric dataset. In specific examples, the sampling kit can include one or more of: a weighing scale (e.g., a connected weighing scale), a fitness tracking device, an ingestible device (e.g., smart pill, gastric balloon, etc.), a wearable computing device, a wearable biometric sensor (e.g., analyte sensor, cardiovascular parameter sensor, respiratory parameter sensor, neurological signal parameter sensor, activity sensor, etc.), or other device configured to facilitate reception of the biometric dataset. Furthermore, the sampling kit can provide instructions or otherwise facilitate linking of any devices provided with the account of the subject.

The biometric dataset of Block S120 can include data sampled from devices once (e.g., at a single time point), or at a number of time points (e.g., at random points, at regular points, in relation to triggering events, with other frequency, etc.), where multiple time points of sampling are described in more detail below with respect to specific examples.

In examples, components of the biometric dataset can be received based upon interactions between subjects and an application executing at mobile devices of subjects, and/or or on a web platform with user accounts for each subject. The application can be structured to retrieve self-reported data from subjects, data reported from caretakers or entities associated with subjects, data retrieved from interfaces (e.g., application programming interfaces, wired or wireless connections) with connected biometric devices associated with subjects, and/or by other methods.

In variations, the methods 100, 200 can include tracking menstruation symptoms, for example, including period start date, period length, days in cycle, current phase, predicted cycle length, and other symptoms/factors.

In variations, the methods 100, 200 can include tracking clinical symptoms, for example, by way of gut health survey data (e.g., derived from GERDq, derived from IBS-SSS, derived from Bristol Stool Chart, etc.), by way of mental health survey data (e.g., derived from diagnostic and statistical manual (DSM) variations, derived from GAD 7, etc.), by way of sleep health survey data (e.g., derived from Insomnia Index Severity, etc.), by way of skin health survey data (e.g., derived from DLQI hair loss data, derived from POEM survey data for eczema, etc.), by way of chronic pain data (e.g., derived from Global Pain Scale data, etc.), and/or by way of other survey data.

In variations, the methods 100, 200, can include tracking biometric data from interfaces with digitally-connected devices, such as through one or more of: Apple™ HealthKit™, Apple Watch™, the Whoop™ platform, the Garmin™ platform, the Google™ health platform, the Fitbit™ platform, the Oura™ platform, the Withings™ platform, the Wahoo™ platform, the Peloton™ platform, the Zwift™ platform, the Training Peaks™ platform, the Polar™ platform, the Suunto™ platform, the Fatsecret™ platform, the FreeStyle Libre™ platform for monitoring blood glucose levels and trends, the Eight Sleep™ platform, the Samsung Health™ platform, the iFit™ platform, the Tempo™ platform, the Concept 2™ platform, the Cranometer™ platform, the Under Armour™ platform, the Renpho™ platform, the Omron™ platform, the Coros™ platform, the Huawei™ platform, the Biostrap™ platform, the Medtronic™ continuous glucose monitor platorm, and/or other suitable platforms and devices for monitoring activity, biometric signals, diet, and/or other information.

The biometric dataset can, however, be received in another manner or using other suitable devices.

### 2.1.3 Lifestyle Aspects

Block S130 recites: receiving a lifestyle dataset from the subject, which functions to enable generation of a baseline state and one or more signatures from which models for returning diagnostics and personalized therapeutic pathways can be generated in subsequent portions of the method.

In variations, the lifestyle dataset captures behavioral information for the subject pertaining to one or more of: energy levels (e.g., morning energy level, evening energy level, daytime energy level, etc.), dietary behavior, sleep behavior, stress levels, stress-associated events, cravings, exercise behavior, meditation behavior, perceived progress toward a health-associated goal, actual progress toward a health-associated goal, state of symptoms, social determinants of health, familial determinants of health, and work determinants of health, and/or other behavioral information. In examples, the lifestyle dataset can capture one or more of the following lifestyle characteristics of the subject: energy levels, food intake (e.g. through food photos which are then assessed and scored by a coaching entity or other entity, through dietary journal entries, through application programming interfaces (APIs) of diet-monitoring applications, etc.), sleep behavior, stress levels, cravings, exercise behavior, and weight loss progress. However, variations of the example can alternatively capture other types of lifestyle information from the subject.

In further examples, the lifestyle dataset can capture one or more of: a morning energy level, dietary behavior, sleep behavior, stress levels, cravings, exercise behavior, meditation behavior, state of symptoms of the subject, medication use, social determinants of health, familial determinants of health, and work determinants of health.

As described above, the sampling kit that facilitates self-sampling of genetic and/or microbiome samples from the subject can further include elements configured to facilitate reception of the lifestyle dataset. In specific examples, the sampling kit can include instructions for or otherwise provide access to a tool for receiving self-report data from the user pertaining to the lifestyle dataset. Additionally or alternatively, the sampling kit can include functionality for linking personal/protected information and/or accounts of the user (e.g., within social media platforms, within health tracking platforms, etc.) with the account of the subject associated with the platform (in a secure manner), such that aspects of the lifestyle dataset can be populated in a more automated manner.

In variations, the components of the lifestyle dataset can be retrieved once (e.g., upon intake of the subject); however, the components of the lifestyle dataset can alternatively be retrieved at a number of time points (e.g., at random points, at regular points, in relation to triggering events, with other frequency, etc.).

In specific examples, demographics and other data retrieved in relation to Block S110, S120, and S130 can include one or more of: demographics (e.g., age, gender, ethnicity, income level, etc.), physical and blood markers (e.g., BMI, previous weight from one year ago, previous weight from two years ago, previous weight from three years ago, waist to hip ratio, fasting glucose, hemoglobin A1c levels, CRP levels, LDL levels, HDL levels, Triglyceride levels, etc.), behavioral and lifestyle aspects (e.g., level and type of exercise, anxiety factors, dietary factors, disease and health history factors, comorbid condition factors), and determinants of health factors (e.g., family size, food budget, pharmaceutical usage history, commute time, nature of work, etc.).

### 2.1.4 Other Relevant Datasets

While types of data are described in Blocks S110-S130, the method 100 can additionally or alternatively include retrieval of other supplemental or contextual data relevant to generating actionable insights in subsequent blocks of the method 100. For instance, the method 100 can include capture of current therapeutic approaches the subject participates in (e.g., existing medications, existing supplements, etc.), trends in usage or adherence to current therapeutic approaches the subject participates in (e.g., increasing use, decreasing use, steady use, etc.), medical history, family medical history, and/or other information. Additionally or alternatively, additional data can include information related to an environment of the user, such as a location of the user (e.g., as determined from a global positioning device, from a triangulation device), environmental temperature of the user, environmental audio of the user, and any other suitable environmental information of the user pertaining to potential stimuli affecting health, and/or environmental devices that can be used for therapeutic outputs associated with subsequent blocks of the method.

### 2.2 Methods - Sample and Data Processing

Block S140 recites: returning a genomic profile, a baseline microbiome state, and a set of signatures (e.g., diagnostic and/or therapeutic signatures associated with genomic and demographic features of the subject) upon processing the set of samples, the biometric dataset, and the lifestyle dataset with a set of transformation operations. Block S140 functions to process samples and transform data from Blocks S110-S130 into various signatures that can be used to characterize the subject and to generate a personalized intervention plan for the subject based upon the characterization of the subject.

In examples, transformation operations can include one or more of: demultiplexing, generating amplicon sequence variants (ASVs), performing taxonomic and functional annotation of the set of sequencing reads based on at least one of local and global alignment methods, and graph-based methods, linear and nonlinear dimensionality reduction, applications of supervised, semi-supervised and unsupervised machine or statistical inference methods to derive informative features from the microbiome profiles, imputing missing sites, determining genetic ancestry of the subject, estimating genetic parameters comprising genetic diversity and homozygosity, estimating scores representing inherited risk, and estimated values of qualitative, continuous and categorical traits which are normalized to biological gender, genetic ancestry, age, socioeconomic status, biometric measurements and life-style variables. Additionally or alternatively, transformation operations can include one or more of: wherein the set of transformation operations further comprises demultiplexing the set of sequencing reads, generating amplicon sequence variants (ASVs) from data derived from the set of sequencing reads, performing taxonomic and functional annotation of data derived from the set of sequencing reads based on alignment methods (e.g., local methods, global methods) and graph-based methods, performing linear and nonlinear dimensionality reductions with data derived from the set of sequencing reads, and performing at least one of machine learning (e.g., supervised, semi-supervised, unsupervised) and statistical inference methods upon data derived from the set of sequencing reads to derive informative features from the baseline microbiome state and/or other microbiome states (e.g., from sample data corresponding to other time points).

Additionally or alternatively, the set of transformation operations can include comprises modeling microbiome data derived from the set of samples as a network, and organizing microbial communities, from the network, into clusters/subgraphs of at least one of (e.g., individually or in combination) amplicon sequence variants, taxa, and pathways, wherein the clusters have intracluster correlations greater than intercluster correlations. In variations, identified microbial clusters/subgraphs from the network can be organized according to one or more characteristics (e.g., descriptors, methods), including 1) a set of ASVs, taxa, or pathways associated with the community or at least 50% of them, 2) a set of keystone ASV, taxa, or pathways, or at least 50% of them, corresponding to the top 20% of elements of the set that account for the majority of intracluster/subgraph connectivity, transitivity, or node-centrality, 3) a continuous variable (e.g., the spectral decomposition of the subgraph or one with a correlation of at least a threshold amount, such as 0.8 the spectral decomposition), 4) a continuous variable (e.g., the spectral decomposition of the abundance matrix of the community members among a set of microbiome samples or one with a correlation of at least 0.8 with the spectral decomposition), or another organization method.

Transformation operations, and architecture and sub-architecture of models are described in further detail below.

### 2.2.1 Sample Processing

In variations, processing the one or more samples can include any one or more of: sample lysis, disruption of membranes, extraction of DNA, RNA, nucleic acid fragments, other nucleic acid material, separation of non-target material from target material(e.g., RNA, proteins) from the sample, nucleic acid purification, nucleic acid fragmentation, nucleic acid amplification, protein extraction, target material washing, target material retrieval, nucleic acid synthesis, such as cDNA synthesis (e.g., from captured mRNA), sequencing of amplified nucleic acids, library preparation, labelling using fluorescent dyes, hybridization to probe sequences contained within arrays, scanning of arrays to assess hybridization intensity between probes and targets, single-base extension of probes with labelled nucleotides, and/or other suitable processing steps. Thus, portions of Block S140 can be implemented using embodiments, variations, and examples of the sample handling network and/or computing system described in further detail.

Amplification can implement suitable amplification techniques (e.g., using polymerase chain reaction (PCR)-based techniques, using helicase-dependent amplification (HDA), using loop mediated isothermal amplification (LAMP), using self-sustained sequence replication (3SR), using nucleic acid sequence based amplification (NASBA), using strand displacement amplification (SDA), using rolling circle amplification (RCA), ligase chain reaction (LCR), etc.)). Amplification can implement primers targeted to specific genetic sequences (e.g., human sequences, non-human sequences), in association with generation of diagnostic and therapeutic signatures. Additionally, amplification can be quantitative (e.g. qPCR) such that amplicons (e.g. human sequence, bacterial sequences, fungal sequences, viral sequences, etc.) can be associated with absolute quantification data. Additionally or alternatively, primers used can be designed to mitigate amplification bias effects, as well as configured to amplify and/or sequence nucleic acids (e.g., of the 16S region, the 18S region, the ITS region, viral genetic markers, full metagenomic sequences, transcriptomic sequences, etc.) that are informative from a microbiome perspective.

In specific examples, nucleic acid sequences of interest include V3-V4 regions of 16S RNA; however, other loci of interest (e.g., housekeeping genes that provide taxonomic and/or functional information, insertions, deletions, etc.) can be assessed. Primers used in variations of Block S140 can additionally or alternatively include incorporated barcode sequences, unique molecule identifiers, adaptor sequences, or other sequences specific to each sample and/or in association with sequencing platforms, which can facilitate identification of material derived from individual samples post-amplification.

Furthermore, sequencing can be performed in coordination with a next generation sequencing platform (e.g., Illumina™ sequencing platform) or other suitable sequencing platform (e.g., Oxford nanopore sequencing platform, PacBio platform, etc.). Additionally or alternatively, any other suitable sequencing platform or method can be used (e.g., a Roche 454 Life Sciences platform, a Life Technologies SOLiD platform, etc.). Additionally or alternatively, sample processing can implement any other step configured to facilitate processing in cooperation with amplification, including adding barcodes for multiplexing, unique molecular identifiers, etc.

Processing the one or more samples can include performing an assessment of single nucleotide polymorphisms (SNPs) of the one or more subjects. SNP array processes can be performed in coordination with a high-throughput genotyping platform (e.g. Illumina™ Infinium Global Screening Array). Additionally or alternatively, any other suitable genotyping platform or method can be used (e.g., an Affymetrix® Genome-Wide Human SNP Array, etc.). Additionally or alternatively, sample processing can implement any other step configured to facilitate processing in cooperation with amplification. In specific examples, performing the assessment of SNPs can include characterizing a panel of genomic SNPs from the one or more subjects, where the panel of SNPs can include more than 2 SNPs, more than 3 SNPs, more than 4 SNPs, more than 5 SNPs, more than 6 SNPs, more than 7 SNPs, more than 8 SNPs, more than 9 SNPs, more than 10 SNPs, more than 11 SNPs, more than 12 SNPs, more than 13 SNPs, more than 14 SNPs, more than 15 SNPs, more than 16 SNPs, more than 17 SNPs, more than 18 SNPs, more than 19 SNPs, more than 20 SNPs, or another suitable number of SNPs.

Sample processing can further include operations for detecting allelic variations (e.g., risk alleles) or alleles of interest from the sample(s) of the subject. In variations, alleles of interest can be bi-allelic or multiallelic. SNPs evaluated can further be characterized by a threshold minor allele fraction (MAF), such as with an MAF above a suitable threshold (e.g., MAF >0.1, MAF >0.2, MAF >0.3, MAF >0.4, MAF >0.5, etc.); however, SNPs evaluated can be characterized with other MAF values. SNPs evaluated can be for coding regions (e.g., synonymous, non-synonymous, missense, nonsense) and/or non-coding regions.

As such, Block S140 can include returning a set of SNP features and a set of microbiome features for each of the set of subjects from the multi-omic model, and generating the personalized intervention plan from the set of SNP features and the set of microbiome features.

Additionally or alternatively, sample processing for generation of genomic data can include operations for detecting other loci of interest from the sample(s) of the subject.

### 2.2.2 Signal Processing

With respect to biometric signal acquisition and/or monitoring devices described above, Block S140 can include signal processing operations including one or more of: denoising, filtering, smoothing, clipping, transformation of discrete data points to continuous functions, and performing any other suitable signal conditioning process. For instance, some variations of Block S140 can additionally include performing a windowing operation and/or performing a signal cleaning operation. Signal cleaning can include removal of signal anomalies by one or more filtering techniques. In specific examples, filtering can include one or more of: Kalman filtering techniques, bootstrap filtering techniques, particle filtering techniques, Markov Chain Monte Carlo filtering techniques; and/or another suitable technique. Signal cleaning can thus improve data quality for further processing, in relation to one or more of: noise, sensor equilibration, sensor drift, environmental effects (e.g., moisture, physical disturbance, etc.), and any other suitable type of signal artifact.

### 2.2.3 Baseline and Signatures

The baseline state (e.g., of genetic factors, of microbiome factors) functions to establish a reference state against which progress is compared, as the subject participates in the personalized intervention plan generated in subsequent portions of the method 100. The baseline state is preferably associated with a state of the subject (e.g., microbiome baseline state) prior to participation in the personalized intervention plan, such that the baseline state characterizes a state of the subject prior to treatment according to the personalized intervention plan. However, the baseline state can alternatively characterize another suitable state of the subject. Furthermore, the method 100 can include re-establishment of a “baseline” state of the subject in coordination with provision of the personalized intervention plan. As such, the baseline state can be updated as the subject progresses during participation in the personalized intervention plan, at which point the diagnostic and therapeutic signatures may lead the subject into a different personalized intervention plan.

The baseline state can also characterize a physiological state of the subject, with respect to a condition or indication (e.g., mental health condition, disease state, etc.) described in more detail below. As such, the baseline state can characterize a clinical diagnosis, a laboratory diagnosis, a radiological diagnosis, a tissue diagnosis, a principal diagnosis, an admitting diagnosis, a differential diagnosis, a prenatal diagnosis, a diagnosis of exclusion, and/or other diagnostic criteria. Additionally or alternatively, the baseline state can characterize aspects associated with a condition or indication (e.g., a state of pain, a state of discomfort, etc.). Additionally or alternatively, the baseline state can characterize aspects associated with an emotional state, state of focus, or other cognitive state of the subject.

In some embodiments, the diagnostic signatures generated in Block S140 can include one or more of: genetic signatures, microbiome signatures (e.g., associated with microbial taxonomic features, associated with microbiome functional features, associated with microbial ecological features, etc.), biometrics, and/or lifestyle biomarkers indicative of decreased or increased likelihood of developing a health condition.

In some embodiments, the therapeutic signatures generated in Block S140 can include one or more of: genetic signatures, microbiome signatures (e.g., associated with microbial taxonomic features, associated with microbiome functional features, etc.), biometrics, and/or lifestyle biomarkers indicative of decreased/increased likelihood of improvement or remission of a health condition. The therapeutic signatures can be used to develop new intervention types, dietary interventions, sleep therapy interventions, pharmaceutical, nutraceutical or skinceutical compositions based on chemistry (e.g., small molecule modulators), biology (e.g., pre-/pro-/syn-/post-biotics), or any other suitable molecular entity. Additionally or alternatively, the therapeutic signatures can be used in Block S150 to generate the personalized intervention plan, with respect to personalized and combinatorial therapeutic pathways implementing different approaches.

### 2.2.3.1 Types of Biomarkers

In variations, genetic biomarkers can include or be derived from one or more of: single and multi-genic single nucleotide polymorphism (SNP)-based biomarkers, tandem repeats, indels (insertions and deletions), somatic amplifications, copy number variations (CNVs), translocations, inversions, other genetic features (e.g., mutations, recombinations, immigrations, etc.), imputed genotypes, genetic ancestry, genetic diversity and homozygosity, genetic scores representing inherited risk, and estimated values of qualitative, continuous and categorical traits which could be normalized to biological gender, genetic ancestry, age, socioeconomic status, biometric measurements and life-style variables, and/or other types of genetic biomarkers.

In examples, the method can include extraction of SNP-based biomarkers (e.g., with risk alleles and variants), which can be detected upon sequencing and/or retrieved from one or more of: genome-wide association studies (GWAS), public repositories and other scientific literature, including allele frequency, odd ratios or beta coefficients for a particular impact on a phenotype, p-values or other statistical values, and study cohort size. Based on these data sources, single gene or multigenic/polygenic trait risk index scores are obtained either by weighted or unweighted scoring (depending on availability of odd ratios or beta coefficients).

Risk indices for each trait are then used for defining interventions. Panels of traits can be configured to include 1 trait (e.g., for a first type of report), 2-4 traits (e.g., for a second type of report), 5-10 traits (e.g., for a third type of report), > 10 traits (e.g., for a fifth type of report). Example reports can include: a nutrition report, a fitness report, an allergy report, a skin health report, a gut health report, a mental health report, a sexual health report, a sleep report, a cardiometabolic report, a hormonal health report, and a musculoskeletal health report, where one or more report types are returned in Block S160 with respect to execution of personalized intervention plans for subjects.

Example traits associated with biomarkers and included as inputs into models for generating personalized intervention plans can include one or more of: zinc needs (e.g., based on SNPs on genes CA1, PPCDC and NBDY), vitamin K needs (e.g., based on SNPs on genes CYP₄F2 and KVKORC1), vitamin E needs (e.g., based on SNPs on genes ZPR1, CYP₄F2, TTPA and CD36), vitamin D needs (e.g., based on SNPs on genes CYP2R1, GC and NADSYN1), vitamin C needs (e.g., based on SNPs on gene SLC₂₃A₁), vitamin B₉ needs (e.g., based on SNPs on genes MYT1L and MTHFR), vitamin B6 needs (e.g., based on SNPs on genes NBPF₃ and ALPL), vitamin B12 needs (e.g., based on SNPs on genes FUT2, CUBN and TCN1), vitamin A needs (e.g., based on SNPs on gene BCMO₁), selenium needs (e.g., based on SNPs on genes DMGDH and CBS), phosphate needs (e.g., based on SNPs on genes RGS1₄ and C12orf₄), magnesium needs (e.g., based on SNPs on genes DCDC₅, PRMT₇, ATP2B1, MUC1, SHROOM₃ and TRPM6), iron needs (e.g., based on SNPs on genes TMPRSS6, TRF2 and TF), cooper needs (e.g., based on SNPs on genes SIMM₁ and SELENBP₁), choline needs (e.g., based on SNPs on genes PEMT and MTHFD1), calcium needs (e.g., based on SNPs on gene CASR ), antioxidant needs (e.g., based on SNPs on genes PON1, CAT, CYP1A2, GSTP1, NQO1, SOD2, NATand GPx1P1), tendency to regain weight (e.g., based on SNPs on genes PPARG, TFAP2B, ADIPOQ and BDNF), tendency to prefer sweet foods (e.g., based on SNPs on genes FGF21, SLC2A2, TAS1R2 and TAS1R3), tendency to prefer fatty foods (e.g., based on SNPs on gene CD36), tendency to prefer bitter foods (e.g., based on SNPs on gene TAS2R38), tendency to overeat (e.g., based on SNPs on genes MC4R, TAS2R38 and FTO), tendency to gain weight (e.g., based on SNPs on genes SH2B1, KCTD15, SEC16B, MC₄R, MTCH2, TMEM18, STK₃₃, ETV₅, BDNF, FTO, NEGR₁ and ADIPOQ), saturated fats intake and weight gain tendency (e.g., based on SNPs on genes FTO and APOA2), protein intake and weight loss tendency (e.g., based on SNPs on gene FTO), poly-unsaturated fats intake and weight gain tendency (e.g., based on SNPs on gene BDNF and FADS), mono unsaturated fats intake and weight gain tendency (e.g., based on SNPs on genes ADIPOQ and PPARG), carbohydrate intake and weight gain tendency (e.g., based on SNPs on genes LRRN6C, FAIM2, FLJ₃₅₇₇₉, FTO, RBJ and SEC16B), salt intake and blood pressure sensitivity (e.g., based on SNPs on genes AGT and NPPA), riboflavin and blood pressure response (e.g., based on SNPs on gene MTHFR), lactose intolerance (e.g., based on SNPs on genes LCT and MCM6), gluten sensitivity (e.g., based on SNPs on genes HLA-DQ2.5 and HLA-DQ8), milk allergy (e.g., based on SNPs on gene TLR6 and IL2), peanut allergies (e.g., based on SNPs on gene HLA-DRA, STAT7 and LOC100507686), histamin intolerance (e.g., based on SNPs on gene AOC1, HNMT and LOC105375567), cockroach allergy (e.g., based on SNPs on gene IL12A), dust mites allergy (e.g., based on SNPs on gene C11orf30, TSLP, GSDMA, IL1RL1 and HLA-DQB₁-AS₁), pets allergy (e.g., based on SNPs on gene HLA-DQB₁), hay fever (e.g., based on SNPs on gene IL1RL1, GSDMA, CLEC16A, WDR36, LRRC32, SMAD₃, ZBTB₁₀, TSLP, IL₃₃ and HLA-DQB₁), pollen allergy (e.g., based on SNPs on gene HLA-DQB₁, TLR₁, IL₁RL₁, GSDMA, TSLP and LRRC₃2), grass allergy (e.g., based on SNPs on gene ABL2, DNAH₅, EPS1₅, LRRC₃2 and genes within the HLA), caffeine metabolism (e.g., based on SNPs on gene CYP1A2), caffeine consumption (e.g., based on SNPs on genes MLXIPL, GCKR, CYP1A2, AHR, ABCG₂ and EFCAB₅), alcohol flush (e.g., based on SNPs on gene ALDH2), weight loss or weight gain with exercise (e.g., based on SNPs on gene FTO ), likelihood of injury (e.g., based on SNPs on genes COL₅A1, MMP₃, ESR1, ACTN₃, MCT1 and IGF2), likelihood of fatigue (e.g., based on SNPs on gene MCT1 ), insulin sensitivity with exercise (e.g., based on SNPs on gene LIPC), HDL cholesterol levels with exercise (e.g., based on SNPs on gene PPARD), exercise recovery (e.g., based on SNPs on gene TNF), exercise motivation (e.g., based on SNPs on gene BDNF), tendon strength (e.g., based on SNPs on genes COL₅A1, COL1A1 and GDF₅), power (e.g., based on SNPs on genes ACTN₃, COTL1, MPRIP, CALCR, MSTN and PPARA), lung capacity (e.g., based on SNPs on genes TTC6 and an intergenic region), ligament strength (e.g., based on SNPs on genes MMP₃ and COL1A1), heart capacity (e.g., based on SNPs on genes MYH6, CD₄6 and KIAA1₇₅₅),hand grip strength (e.g., based on SNPs on genes GLIS1, HOXB₃, Dec1, GBF1, MGMT, SYT1, HLA, KANSL1 and TGFA), flexibility (e.g., based on SNPs on gene COL₅A1 ), endurance (e.g., based on SNPs on genes BDKRB2, ACTN₃, ADRB₃, AMPD1 and IL6), aerobic capacity (e.g., based on SNPs on genes NFIA-AS2, TSHR, ESRRB, PPARA and CREB1), and/or other traits.

Panels of SNPs implemented as model inputs can include SNPs describe in relation to specific health conditions below. Panels of SNPs can additionally or alternatively include other SNPs, risk alleles and/or other loci of interest, associated with other genes of interest, in relation to the genomic profiles of subjects.

SNPs can include SNPs having a desired minor allele fraction (MAF) for discrimination between non-risk and risk alleles. The MAF can be greater than 0.2, 0.3, 0.4, 0.5, 0.6, 0.7, 0.8, or 0.9. SNPs evaluated can be for coding regions (e.g., synonymous, non-synonymous, missense, nonsense) and/or non-coding regions. SNPs evaluated can be biallelic or multiallelic, with more than two alleles per SNP. Furthermore, SNPs selected for evaluation can have allele pairs that are well-discriminated (e.g., with respect to stabilization or destabilization characteristics). For instance, SNPs can be selected with prioritization of G/T, C/A, and T/A SNPs having high destabilization strength characteristics. The size of the SNP panel being evaluated, threshold MAF for each SNP, and distribution can thus be selected to optimize or otherwise increase the probability of returning an accurate model output in subsequent portions of the embodiments, variations, and examples of the method 100 described. In examples described below, trained models can return output combinations of SNP biomarkers having high predictive power in relation to one or more of: mental health conditions (e.g., anxiety, depression, sleep, etc.), weight-related conditions (e.g., associated with BMI), comorbid conditions, and/or other conditions.

In variations, microbiome biomarkers can be derived based on annotation methods that utilize one of local and global sequence alignment, graph-based (network) methods, linear and non-linear dimensionality reduction, and applications of supervised, semi-supervised and unsupervised machine or statistical inference methods using compositionally transformed and arithmetically transformed values from microbial taxonomic and functional abundances and can include one or more of: taxonomic features (e.g., based on operational taxonomic units (OTUs), based on amplicon sequence variants (ASVs), etc.); probiotic features (e.g., single strains associated with health outcomes), consortia biomarkers (e.g., derived from leveraging artificial intelligence to identify bidirectional links between bacteria in human ecological niches, in order to understand which microorganisms co-occur with or do not co-occur with others to define consortia, etc.); prebiotic biomarkers (e.g., specific fiber types, functionality of specific fibers with respect to taxonomies represented in the gut microbiome, etc.); synbiotic features (e.g., combinations of pro- and pre-biotic features, etc.); microbiome functional features, (e.g., postbiotic biomarkers, such as proteins or metabolites from microorganisms, other metabolic features, other functional features, etc.); and/or other types of microbiome-associated biomarkers.

In examples, microbiome biomarkers can include one or more of: a richness diversity index, a Shannon diversity index, a phylogenetic entropy index, beta diversity indices (e.g. weighted and unweighted UniFrac distances, bray-curtis dissimilarity index etc.), relative abundances of taxonomic groups identified, acetate metabolism relative abundance, propionate metabolism relative abundance, butyrate metabolism relative abundance, polyamine metabolism relative abundance, serotonin synthesis relative abundance, GABA synthesis relative abundance, vitamin B1 (thiamine) synthesis relative abundance, vitamin B2 (riboflavin) synthesis relative abundance, vitamin B₃ (niacin) synthesis relative abundance, vitamin B₅ (pantothenate) synthesis relative abundance, vitamin B6 (pyridoxine) synthesis relative abundance, vitamin B₇ (biotin) synthesis relative abundance, vitamin B₉ (folate) synthesis relative abundance, vitamin B12 (cobalamin) synthesis relative abundance, vitamin C synthesis relative abundance, vitamin K synthesis relative abundance, lactose degradation relative abundance, gluten degradation relative abundance, caffeine metabolism relative abundance, alcohol metabolism relative abundance, trimethylamine N-oxide synthesis relative abundance, histamine synthesis relative abundance, and/or other microbiome biomarkers. Additionally or alternatively, the microbiome markers can include abundance (e.g., absolute, relative or differential) of keystone microbial species and functional pathways as identified by machine learning algorithms, associated with health and disease conditions (e.g. gut health, mental health, metabolic disorders etc.).

Additional examples of microbiome taxa and/or functions are described in more detail in Sections 2.5.1 and 2.5.2 below.

In examples described below, trained models can return output combinations of microbiome-associated biomarkers having high predictive power in relation to one or more of: mental health conditions, comorbid conditions, weight-related conditions and/or other conditions.

In variations, lifestyle biomarkers can include or be derived from one or more of: energy levels (e.g., morning energy levels, midday energy levels, evening energy levels, etc.), dietary behavior (e.g., consumption behavior), sleep behavior, stress levels, stress-associated events, cravings, exercise behavior, perceived progress toward a health-associated goal, actual progress toward a health-associated goal (e.g., weight loss progress, fitness progress, exercise regimen progress, etc.), changes in appetite, state of symptoms, and/or other lifestyle biomarkers.

In variations, therapeutics biomarkers can include or be derived from one or more of: medication usage (e.g., current usage, changes in usage over time), supplement usage (e.g., current usage, changes in usage over time), psycho-social intervention biomarkers (e.g., cognitive behavioral therapy (CBT) characterizations), and/or other biomarkers associated with existing therapeutics that the subject interacts with.

Model outputs can be processed in relation to input subject condition statuses, subject body mass index (BMI) at different time points (e.g., in embodiments where the method is performed iteratively), subject gender, combinatorial inputs (e.g., condition status: BMI and condition status:gender), residuals, and/or other inputs described above.

### 2.2.4 Model Architecture

In embodiments, models for processing inputs described and returning outputs described, in order to achieve the performance characteristics described, can include statistical models structured to receive input data, and to return indications of subsets of features having high predictive power with respect to subject characterization (e.g., in terms of estimates, in terms of standard error, in terms of t-values, in terms of P-values, etc.).

In variations, model architecture can include regression models (e.g., linear regression models, logistic regression models, polynomial regression models), correlation models (e.g., Pearson correlation, Kendall rank correlation, Spearman correlation, Point-Biserial correlation, etc.), and/or other model architecture. In specific examples, regression models can include quantile regression architecture (e.g., for data with high levels of outliers, high skewness and heteroscedasticity), ridge regression architecture, lasso regression architecture, elastic net regression architecture, principal components regression, partial least squares regression architecture, support vector regression, ordinal regression architecture, Poisson regression architecture, negative binomial regression architecture, quasi Poisson regression architecture, Cox regression architecture, Tobit regression architecture, and/or other regression architecture, with or without regularization and/or implementation of loss functions.

Prior to fitting models, input data can be conditioned or otherwise preprocessed, such that conditioned data elements (e.g., genomic reads associated with SNPs, risk alleles, variants, and/or other loci of interest, microbiome reads, microbiome functional data, behavioral data, sensor data, other contextual data described above, etc.) are suitable for further processing. Conditioning, as described above, can include filtering of data (e.g., removing sequencing reads or sensor data outputs that have confidence values below a threshold, removing sequencing reads having missing values greater than a threshold level, etc.).

In variations, the method 100 can include transforming output data into digital objects, such as visualizations, in order to facilitate generation of characterizations, interventions for condition prevention, interventions for condition treatment, and/or other actions (e.g., computer-generated and machine-implementable instructions) for improving or maintaining subject health. In specific examples, digital objects and visualizations can be generated upon transforming input data, using various statistical packages (e.g., R stats, ggplot2, pscl, car, pROC, Metrics, caret, glmnet, tidyverse, lubridate, imputeTS, and ggpubr packages) in sequence and/or in parallel.

Specific implementations of model architecture for health condition areas are described in Section 2.5 below, and embodiments, variations, and examples of model training are described in Section 2.6 below.

### 2.3 Methods - Exemplary Conditions Targeted

In variations, biomarker signatures and baseline states, as well as personalized therapeutics developed using the invention(s) described, can target one or more health conditions or indications.

In examples, conditions targeted can include one or more of: mental health conditions (e.g., anxiety, depression, autism, bipolar disorder, memory loss, brain fog, cognitive functions etc.); sleep issues (e.g., sleep apnea, disturbed sleep, fatigue, etc.); obesity and other weight-associated conditions; acute pain, chronic pain associated with other conditions (e.g., joint pain including persistent joint aches and joint swellings, osteoarthritis, gout, rheumatoid arthritis, etc.); and/or other conditions.

### 2.4 Methods - Personalized Intervention Plan

Block S150 recites: generating a personalized intervention plan for the subject upon processing the genomic profile, the baseline microbiome state, and the set of signatures (e.g., diagnostic signatures, therapeutic signatures) with a multi-omic model, which functions to transform signatures into a customized combination of therapeutic approaches tailored to improve or maintain a health state of the subject.

The multi-omic model is preferably constructed to process signatures and/or baseline states generated from Block S140, and to return the personalized intervention plan that is tailored to the subject. In variations, the multi-omic model can return a set of SNP features and a set of microbiome features for the subjects, and generate their personalized intervention plans from the set of SNP features and the set of microbiome features.

In variations, the personalized intervention plan can have subportions (e.g., modules, phases). In variations, the subportions can include portions including one or more of: personalized medication regimens (e.g., based upon pharmacogenomics outputs generated from detected SNPs and microbiome factors for the subjects), personalized supplement regimens (e.g., based upon outputs generated from detected SNPs and microbiome factors for the subjects), personalized diets (e.g., based upon outputs generated from detected SNPs and microbiome factors for the subjects), personalized exercise regimens, personalized sleep regimens, personalized lifestyle recommendations, personalized behavioral therapeutic approaches (e.g., cognitive behavioral therapeutic approaches, etc.), medical procedures, preventative healthcare therapeutic approaches, and/or other suitable aspects. Furthermore, the personalized intervention plan can be characterized with a duration, such that the subject can complete the program and achieve one or more health goals. As such, generating the personalized intervention plan can include returning recommendations (e.g., dietary recommendations, sleep regimen recommendations, exercise regimen recommendations, etc.) and coaching components delivered in person and digitally, configured to adjust taxonomic abundances and microbiome function represented by microorganism taxa of the set of microbiome features, based upon risk alleles of the set of SNP features of the subject. However, the personalized intervention plan can be otherwise configured.

In variations, Block S150 can include generation of instructions, that can be executed by systems having a computing element (e.g., as described in more detail below). For instance, one or more components of the personalized intervention plan can be delivered digitally through a mobile device application, by way of an application-interface between coaching entities and the subjects being treated.

In one example, the personalized intervention plan can have a set duration (e.g., 36 weeks, 24 weeks, 12 weeks, another suitable number of weeks, etc.), and can be configured for delivery using one or more interfaces (e.g., web interface, mobile device interface, wearable computing device interface, telephonic interface, in-person interface, interface with one or more robotic devices, etc.) with the subject.

The example personalized intervention plan has a first phase, a second phase, and a third phase configured to promote behavior change in the subject, provide therapeutic interventions, and achieve desired outcomes with respect to diagnoses and characterizations generated (e.g., based on diagnostic signatures processed by the model, based on therapeutic signatures processed by the model, etc.) in prior blocks of the method 100.

In the example, the first phase has a duration (e.g., 4 weeks, another suitable number of weeks) configured to guide the subject in focusing on baselining health and habits, with functionality for tracking lifestyle through a mobile device application. In the example, the second phase has a duration (e.g., 12 weeks, another suitable number of weeks) focused on personalizing therapeutic approaches for producing transformations in behavior for the subject. In the example, the third phase has a duration (e.g., 8 weeks, another suitable number of weeks) focused on personalizing therapeutic approaches for stabilizing the subject’s life, with respect to maintenance of desired states (e.g., building and maintaining healthy habits, preventing relapse, maintaining remission, etc.).

In more detail with respect to the personalized intervention plan, the platform can provide the kit (e.g., genetic and microbiome sampling kit, connected devices, biometric devices, instructions, etc.), and interaction with the kit by the subject facilitates establishment of a physiological baseline that can serve as a reference point for progress, with respect to the personalized intervention plan. In the example, the kit can be mailed (e.g., as facilitated by the platform), to the subject’s home. The kit can be provided through a healthcare provider, in coordination with insurance. The kit also provides instructions for downloading an application with an interface to the platform, as well as functionality for receiving inputs associated with the lifestyle dataset described above. In one specific example, the subject is prompted to download the mobile application, complete a lifestyle intake form, and schedule a counseling session as part of a personalized therapeutic approach.

Subsequently, with respect to the personalized intervention plan, the platform guides and supports the subject using counseling entities (e.g., specialized counselors, artificial intelligence counseling entities, etc.) through a multi-week chronic lifestyle illness transformation journey with regular sessions (e.g., weekly and bi-weekly sessions) that can be scheduled and/or ad hoc. In more detail, a counseling session of the specific example can be a 15-20 telephonic behavioral counseling session, but variations of the example can have another duration and/or be provided in another format. The personalized intervention plan provides personalized therapeutic pathways configured to reconfigure diet, lifestyle and physical activity of the subject to achieve a goal (e.g., 5-10% weight loss goal, 1 BMI unit reduction, 2 BMI unit reduction, 3 BMI unit reduction, 4 BMI unit reduction, 5 BMI unit reduction or other BMI unit reduction, other goal) in combination with other reductions in symptom severity described, which is shown to reduce chronic inflammatory conditions. With respect to the personalized intervention plan generated in Block S150, personalized and actionable insights are returned by the model and delivered to the subject (e.g., through entities, through application interfaces, through other interfaces with the platform, etc.) as they complete phases of the personalized intervention plan (e.g., complete tasks, interact with modules, interact with tools for monitoring diet (e.g., through upload of photo documentation of their meals/diet/consumption), monitor their lifestyle vitals: morning energy, sleep, stress, cravings, exercise, and weight loss progress through the mobile application and wireless scale, etc.). In examples, the subject can engage with the personalized intervention plan at any time or place via their mobile device application and/or web application, which allows for flexible participation. Additionally or alternatively, the personalized intervention plan can provide time-sensitive tasks and/or prompt interaction in a timely manner, with respect to more acute states of the subject (e.g., triggering events, symptom flare ups, etc.).

In variations, the personalized intervention plan can further provide one or more of the following: a report of behavioral traits (e.g., including a description of hormones and enzymes combined with underlying genetic factors that shape mental health issues including anxiety, sleep, and depression); a report of dietary recommendations for adjusting hormones and enzymes that can affect health status (e.g., with exemplary modulation of tryptophan, serotonin, acetate, butyrate, propionate, and others in relation to modulation of mental health state); a report of genetic and microbiome factors of the subject in relation to specific hormones and enzymes; and other information. In examples, the personalized intervention plan can include a dietary regimen customized to the user based upon outputs of Block S140, where probiotics can be included to modulate tryptophan levels, tryptophan-rich foods and vitamin B-rich foods can be included to modulate serotonin levels, prebiotics can be included to modulate acetate levels, and other consumables can be included to modulate levels of other hormones and enzymes, specific to the state of the user. In examples, a subject can be provided with a summary of genetic markers and microbiome features that indicate lower levels of a hormone (e.g., plasma serotonin) in relation to a cohort with similar demographics, as well as functions of serotonin in relation to mental health statuses of the subject.

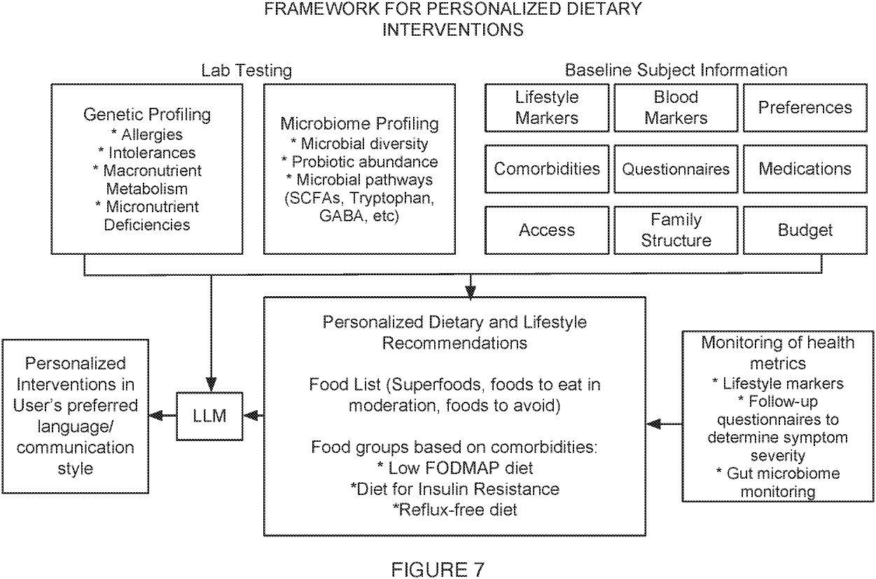

In variations, generating the personalized intervention plan for a subject can implement large language models (LLMs) based upon personalized traits of each subject. An exemplary framework for dietary interventions is shown in FIG. 7, where outputs of blocks S110 through S140 with genetic profiling (e.g., identifying allergies, intolerances, macronutrient metabolism, micronutrient deficiencies, etc.), microbiome profiling (e.g., identifying microbial diversity, probiotic abundance, microbial pathways involving SCFAs, Tryptophan, GABA, etc.), and subject profiling (e.g., with lifestyle markers, blood markers, preferences, comorbidities, clinical questionnaires, medications, access to care factors, family structure, socioeconomic factors, etc.) were processed as inputs by trained models. Returned outputs corresponding to personalized intervention plan components included: dietary and lifestyle recommendations, including food lists (e.g., superfoods, foods to eat in moderation, foods to avoid, foods based upon comorbidities, related to low fodmap diets, diets for insulin resistance, reflux free diets, etc.), monitoring of health metrics (e.g., lifestyle markers, clinical questionnaire updates, microbiome monitoring) to generate feedback loops for refinement of trained models), and implementation of large language models for providing personalized intervention to subjects in their preferred languages for improved adherence.

In variations, the personalized intervention plan can additionally or alternatively provide one or more of the following: inflammatory nutrition and fitness insights; probiotic analysis and functional gut microbiome reports; food recommendations (e.g. meal plans to adjust abundances of various microorganisms); meal plans to decrease pathogenic or pathobiont microorganisms; meal plans to modulate specific microbial functions; etc.); prebiotic recommendations; gut biome monitoring to track immunity and gut microbiome-related disorders; other microbiome characteristics to track other statuses and conditions; and other information.

In examples, model outputs can be used to evolve a food guide of the personalized intervention plan, as the subject progresses through the phases of the program and updates when a new set of information about the subject is available. In an example, the food guide contains at any given time: a food list, foods to eat, foods to exclude, foods to eat in moderation, a sample meal plan, a grocery list, a home food inventory list with functionality for updating the inventory based upon consumption by the subject, portion guidelines, how to structure an approved meal plan, any other information about the current stage of the subject journey, ancillary food guides for major food chains and generic recommendations common to all phases (e.g., approved snacks, tips for craving, tips for takeout food).

In the example, in the first phase the food guide can be personalized based on preferences, exclusions and comorbidities informed by subjects in intake forms. In the second phase, the guide can be further personalized incorporating information about deficiencies, intolerances and high risks derived from genetic traits in the DNA nutrition report and DNA fitness report, as well as microbial diversity, probiotic abundance and microbial metabolic pathway abundance from the gut microbiome report. In the third phase, the guide can be further personalized incorporating information about additional deficiencies, intolerances and high risks derived from additional genetic traits in the DNA reports (e.g., allergy report, skin health report, gut health report, mental health report, sexual health report, sleep report, cardiometabolic report, hormonal health report, and musculoskeletal health report), as well as comparative features in two or more gut microbiome reports, including comparative diversity index, comparative probiotic abundance, and comparative microbial metabolic pathways.

In variations, generating and/or executing the personalized intervention plan (e.g., as in Block S160) can include automatic ordering (e.g., with approval by the subject) of food items through meal, food, and/or grocery-delivery applications (e.g., web applications, mobile device applications, etc.), such as Doordash™, Uber Eats™, and other applications, in order to facilitate adherence to dietary regimens by subjects.

In another example, a specific meal plan of a personalized intervention plan to adjust abundances of a specific microorganism (or profile of microorganisms). For instance, meal plans can be generated based on model outputs, and foods for promoting certain bacteria can be recommended to subjects that have low abundances (e.g., of Akkermancia muciniphila) in their gut microbiome samples when compared to a healthy reference cohort, after a low fermentable oligosaccharides, disaccharides, monosaccharides and polyols (FODMAP) diet, after completing an antibiotic course, or when hitting a weight plateau. The meal plan of the specific example can incorporate a grocery list highlighting protein, fruit and vegetables; a home food inventory list including broth, dairy, fermented dairy, nuts & seeds, prebiotic, salad dressings, seasonings, spices; breakfast recipes, lunch recipes, and dinner recipes; a how to plan your own meal plan section; and a food list including protein, fats, nuts, seeds, group 1 vegetables, fermented dairy, group 2 vegetables, fermented vegetables, other fermented products, group 3 vegetables, prebiotics, beverages, herbs & spices, group 1 fruits, and group 2 fruits.

In an additional example, the personalized intervention plan provides prebiotic recommendations specific to tackle deficiencies or health conditions. A first blend containing chicory inulin powder and partially hydrolyzed guar gum is recommended to subjects suffering from a group of health conditions (e.g., constipation, and those seeking to lose weight with no additional gut health comorbidities). A second blend containing chicory inulin powder and 100% green bananas is recommended to subjects suffering from a different group of health conditions (e.g., diarrhea). Recommended dosage is from 2.5 g to 50 g per day for intervals of up to 60 days for subjects intending to reduce weight, but who suffer from other comorbid health conditions.

In an additional example, a personalized intervention plan included provision of a prebiotic blend of organic inulin (e.g. from chicory) with flavonoids, withanolides, sitoindosides and acylsterylglucosides (e.g from Ashwagandha [Withania somnifera]), along with the following instructions for use: 1) start with 1 tsp per day and increase to 5 tsp per day over 3 - 4 weeks, depending on tolerance level; 2) distribute it across different meals instead of intaking it in one go to maximize the benefits; 3) add to smoothies, shakes, yogurt, soups, water, milk, and plant milk. Use a blender or frother for best results; 4) initially may cause bloating, gassiness, and in rare cases, cramps; 5) continue eating fiber in your meals and prioritize an ND score of 12-14; 6) avoid taking prebiotics during the fasting window. As such, the personalized intervention plan can include provision of a prebiotic blend of chicory inulin and Ashwagandha (Withania somnifera), according to a regimen of consumption.

In an additional example, personalized intervention plans included recommendations, prescriptions for, or provision (through medication channels) of medications according to pharmacogenomic characteristics of subjects identified in prior steps of the methods 100, 200. Exemplary medications included antidepressants and other psychoactive drugs, where genetic changes affecting CYP enzymes that work on the liver were linked to changes in drug responses to certain antidepressants and other psychoactive drugs. As such, based upon trained model outputs specific to each subject, the following recommendations/medications can be provided: a) based on CYP2C19 detection or changes, Citalopram, Escitalopram, Sertraline, Amitriptyline, Clomipramine, Doxepin, Imipramine, Trimipramine, and other medications; b) based on CYP2B6 changes, Bupropion and other medications; c) based on CYP2C9 detection or changes, Phenytoin. In one embodiment, the methods 100, 200, can include: based on detection of gene CYP2C19 from the genomic SNP profile, the personalized intervention plan provides pharmacogenomics information for Citalopram, Escitalopram, Sertraline, Amitriptyline, Clomipramine, Doxepin, Imipramine, and Trimipramine; based on detection of gene CYP2B6 from the genomic SNP profile, the personalized intervention plan provides pharmacogenomics information for Bupropion; and based on detection of gene CYP2C9 from the genomic SNP profile, the personalized intervention plan provides pharmacogenomics information for Phenytoin.

In variations, the application environment (e.g., mobile application environment, web application environment, etc.) can support one or more of the following: a dietary consumption log (e.g., food log, drink log, etc.) that can receive inputs from the subject and/or automatically track consumption of the subject (e.g., in coordination with applications supported by Apple Health™, Google Health™, etc.); a prebiotic consumption log; a Bristol stool scale log; personalized meal and fitness plans generated from the personalized intervention plan; personalized guidance for anxiety, stress and sleep management generated from the personalized intervention plan. Additionally or alternatively, the application environment can include interfaces for connections with (e.g., using Bluetooth™, using another protocol) connected smart devices (e.g., as described above, for automatic weight, cardiovascular parameter tracking, blood analyte parameter tracking, motion tracking, etc.).

In variations, the application environment can further support telehealth interactions between the subject and a counseling/healthcare-providing entity. For instance, the application environment can provide one or more of: communication interfaces that connect the subject with a physician and/or facilitate delivery of care to the subject (e.g., through automated processing of insurance claims, through generation of appointments, through enabling consultations with a medical doctor, etc.); communication interfaces that provide constant or near constant access (e.g., 24 hour, 7 days a week access, etc.) to trained coaches, nutritionists, counselors, therapists, etc.; communication interfaces that enable telehealth group coaching; exercise regimen components (e.g., group fitness content, exercise guidance content, such as yoga content, etc.); stress management material (e.g., provided to the subject in response to detected stress states and/or triggering events, provided to the subject such that the subject can access the content in a convenient manner, etc.); interfaces to a private (e.g., invite-only) social network or community; tasks (e.g., healthy habits challenges); interfaces for reward provision (e.g., community celebration events, incentives, other perks, etc.); and other suitable interfaces.

The application environment can, however, support other suitable functionality associated with the personalized intervention plan.

### 2.5 Methods - Execution of Personalized Intervention Plan and Exemplary Results

Block S160 recites: executing the personalized intervention plan for the subject, where executing the personalized intervention plan can involve executing components of the intervention plan through interfaces described above and/or in relation to the system described below. As such, execution can involve mobile device application interfaces, web application interfaces, interfaces with an entity (e.g., human care-providing entity, digital care-providing entity, etc.), and/or other suitable interfaces.

Execution of the personalized intervention plan produces improved outcomes for subjects participating in their respective personalized intervention plans, examples of which include: improved engagement (e.g., enrollment of over 90% of participants; consistent engagement by a significant percentage, such as 80% of participants after 60 days); improved outcomes (e.g., significant improved weight loss, significant reductions in mental health condition symptoms associated with anxiety symptoms, depression symptoms, sleep symptoms, pain symptoms, etc.); reduction in medication use/necessity; reduction in healthcare costs; and other suitable benefits.

Additionally or alternatively, improved outcomes can be measured at an individual level or at an aggregate number of subjects within a cohort and can include: decreased brain fog or memory challenges symptomatology, decreased depression symptomatology, decreased disturbed sleep symptomatology, decreased headaches or migraines symptomatology, decreased insomnia symptomatology, decreased sleep apnea symptomatology, overall mental health issues symptoms improvement, overall sleep-related conditions symptoms improvement, decreased chronic pain symptomatology, decreased numbers of instances of suicide ideation, decreased anxiety symptomatology, and other improved outcomes.

Additionally or alternatively, improved outcomes can be measured at an individual level or at an aggregate number of subjects within a cohort and can include: percent weight loss, number of units of BMI reduced, weight loss over a period of time, average number of months maintaining weight loss after completing the program, average reduction in HbA1C levels, average reduction of fasting blood glucose, and other improved outcomes.

Executing the personalized intervention plan in Block S160 can include providing results presented in a genetics section of one or more reports generated from model outputs (e.g., within an application environment), which were determined by the number of markers and risk genotypes present in the genomic raw data. Reports can then be transmitted to the entities involved (e.g., subjects, caretakers, insurance companies, etc.) by mobile application and/or web application architecture. Executing the personalized intervention plan in Block S160 can further implement genetic risk profiles to guide the course of subjects’ precision care, as well as analyzing gut microbiome profiles (collected from regular stool swab sampling) to guide the course of care. Based on analysis of these genetic and gut microbiome risk profiles, an analysis (e.g., a summary report, such as one or more of the summary reports described above) can thus be provided in Block S160, and in an exemplary program implementation, the results were systematically reviewed with the participants 1:1 by the health coach over a 4-month period at regular, pre-determined, weekly and bi-weekly intervals.

Executing the personalized intervention plan in Block S160 can further include providing a personalized care program that implements body metrics, gut microbiome and genetic profiles, and personalized health-coaching to manage weight loss. In accordance with the personalized care program, participants can be provided with digital tools for tracking lifestyle and wellness markers (i.e., weight, sleep, hunger, cravings, stress, meditation, superfoods, energy, foods to avoid, foods to eat in moderation, and exercise), documenting the food they consume (e.g., through a photo journal, through a text journal, through a wearable device, etc.), and are assigned a health coach who works personally with the participant through guided sessions as scheduled by the subjects to interpret the personalized wellness reports generated from participants’ app usage and from sampling participants’ DNA and gut microbiota. In accordance with the personalized care program, the reports also provide a breakdown of obesity risk based on individuals’ genetic and gut microbiome profiles. The program can be geared toward participant goals.

To achieve this goal, example implementations of the program were structured to provide automated and manual tools for motivating participants to make incremental lifestyle changes focused around reducing consumption (e.g., of sugars, etc.), timing meals to optimize metabolic processing, reducing systemic inflammation by identifying possibly inflammatory and anti-inflammatory nutrients by genetic testing, establishing a base level of physical activity in a manner that reduces inflammation and that aligns with periods of higher energy and motivation of the subject(s), optimizing gut health based on microbiome testing, and establishing behavioral modifications from the personalized intervention plan as habits, supported by health coaching and application interfaces, such that the changes are sustainable long-term.

In specific examples, implementation of the personalized intervention plan, based upon outputs of models described, achieved groundbreaking performance with respect to individual and/or simultaneous reduction in symptom severity associated with one or more of: mental health condition symptoms (e.g., of anxiety, of depression, of sleep-related disorders), comorbidities (e.g., associated with weight gain, associated with chronic inflammatory pain, associated with gastrointestinal health condition symptoms, associated with cardiovascular health condition symptoms, associated with skin conditions symptoms, associated with other symptoms, etc.).

Variations of the methods described can produce over a 40% reduction in mental health condition symptom severity, over a 41% reduction in mental health condition symptom severity, over a 42% reduction in mental health condition symptom severity, over a 43% reduction in mental health condition symptom severity, over a 44% reduction in mental health condition symptom severity, over a 45% reduction in mental health condition symptom severity, over a 46% reduction in mental health condition symptom severity, over a 47% reduction in mental health condition symptom severity, over a 48% reduction in mental health condition symptom severity, over a 49% reduction in mental health condition symptom severity, over a 50% reduction in mental health condition symptom severity, over a 51% reduction in mental health condition symptom severity, over a 53% reduction in mental health condition symptom severity, over a 42% reduction in mental health condition symptom severity, over a 54% reduction in mental health condition symptom severity, over a 55% reduction in mental health condition symptom severity, over a 56% reduction in mental health condition symptom severity, over a 57% reduction in mental health condition symptom severity, over a 58% reduction in mental health condition symptom severity, over a 59% reduction in mental health condition symptom severity, over a 60% reduction in mental health condition symptom severity, over a 61% reduction in mental health condition symptom severity, over a 62% reduction in mental health condition symptom severity, over a 63% reduction in mental health condition symptom severity, over a 64% reduction in mental health condition symptom severity, over a 65% reduction in mental health condition symptom severity, over a 66% reduction in mental health condition symptom severity, over a 67% reduction in mental health condition symptom severity, or greater reduction in symptom severity, for an individual or across a population of subjects (e.g., on average), in relation to severity prior to receiving the personalized intervention plan.

Additionally or alternatively, variations of the methods described can produce over 15% reduction in depression/anxiety symptoms, produce over 20% reduction in depression/anxiety symptoms produce over 30% reduction in depression/anxiety symptoms, over 40% reduction in depression/anxiety symptoms, over 50% reduction in depression/anxiety symptoms, over 60% reduction in depression/anxiety symptoms, over 70% reduction in depression/anxiety symptoms, over 80% reduction in depression/anxiety symptoms, over 90% reduction in depression/anxiety symptoms, over 95% reduction in depression/anxiety symptoms, over 99% reduction in depression/anxiety symptoms, or greater values of reduction in depression/anxiety symptoms in comparison to baseline states for a subject on the personalized intervention plan.

Variations of the methods described can produce over 15% reduction in insomnia/sleep disorder symptoms, 20% reduction in insomnia/sleep disorder symptoms, produce over 30% reduction in insomnia/sleep disorder symptoms, produce over 40% reduction in insomnia/sleep disorder symptoms, 50% reduction in insomnia/sleep disorder symptoms (e.g., apnea, disturbed sleep), over 60% reduction in insomnia/sleep disorder symptoms, over 70% reduction in insomnia/sleep disorder symptoms, over 80% reduction in insomnia/sleep disorder symptoms, over 90% reduction in insomnia/sleep disorder symptoms, over 95% reduction in insomnia/sleep disorder symptoms, over 99% reduction in insomnia/sleep disorder symptoms, or greater values of reduction in insomnia/sleep disorder symptoms in comparison to baseline states for a subject on the personalized intervention plan.

Variations of the methods described can produce over 1% more weight loss, over 2% more weight loss, over 3% more weight loss, over 4% more weight loss, over 5% more weight loss, over 6% more weight loss, over 7% more weight loss, over 8% more weight loss, over 9% more weight loss, over 10% more weight loss, over 15% more weight loss, over 20% more weight loss, over 25% more weight loss, or greater weight loss in comparison to baseline states for a subject on the personalized intervention plan. Alternatively, variations of the methods described can produce a reduction of 0.5 BMI units, a reduction of 1 BMI units, a reduction of 1.5 BMI units, a reduction of 2 BMI units, a reduction of 2.5 BMI units, a reduction of 3 BMI units, a reduction of 4 BMI units, a reduction of 5 BMI units, a reduction of 6 BMI units, or more, for an individual or across a population of subjects (e.g., on average), in relation to severity prior to receiving the personalized intervention plan.

Variations of the methods described can produce over 40% reduction in brain fog/memory challenge symptoms, over 50% reduction in brain fog/memory challenge symptoms, over 60% reduction in brain fog/memory challenge symptoms, over 70% reduction in brain fog/memory challenge symptoms, over 80% reduction in brain fog/memory challenge symptoms, over 90% reduction in brain fog/memory challenge symptoms, over 95% reduction in brain fog/memory challenge symptoms, over 99% reduction in brain fog/memory challenge symptoms, or greater values of reduction in brain fog/memory challenge symptoms in comparison to baseline states for a subject on the personalized intervention plan.

Severity in various symptoms can be determined at multiple time points, prior to, during, and/or after receiving the personalized intervention plan. Severity can be determined according to the methods 100, 200 described, by way of survey data (e.g., from survey tools described) that is self-reported by subjects or reported by an entity in communication with a subject, biometric monitoring and/or mobile device-based monitoring (e.g., to determine sleep behavior, to determine communication behavior through application programming interfaces with communication applications executing on the mobile device of a subject, to determine activity behavior with sensors and/or calendar applications, to determine other behavior, etc.).

Variations of the methods described can produce one or more of the above results simultaneously, within a duration of 2 days, 3 days, 4 days, 5 days, 6 days, 7 days, 8 days, 9 days, 10 days, 20 days, 30 days, 40 days, 50 days, 60 days, 70 days, 80 days, 90 days, 100 days, any intermediate number of days, or greater than 100 days, with sustained maintenance of results.

Embodiments, variations, and examples of the methods described can simultaneously reduce severity of symptoms and/or increase weight loss with the personalized care program components generated based upon model outputs.

Variations of the methods described can produce other reductions in symptom severity, for other symptoms as well. Exemplary methodologies, implementations of personalized care plans, and performance benefits over current care options are described as follows:

### 2.5.1 Method Exemplary Execution and Performance - Mental Health Conditions

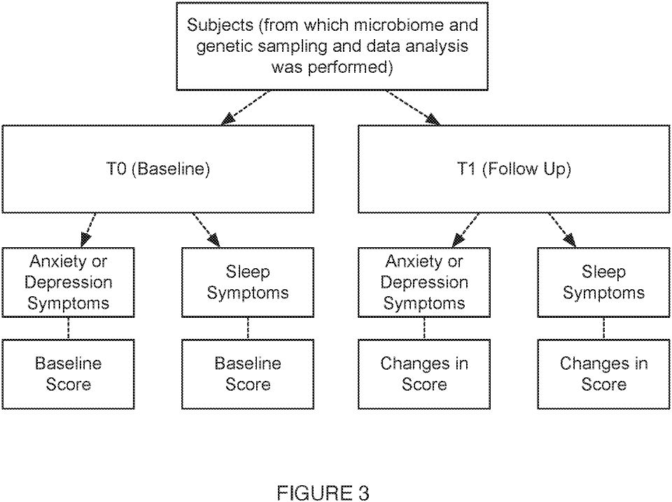

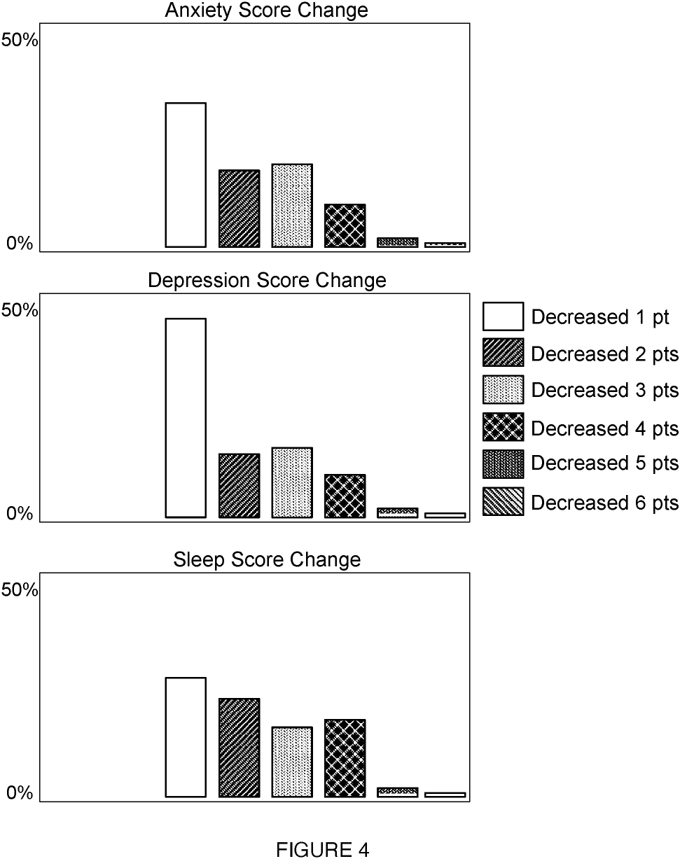

In an exemplary application of the platform for prevention and/or treatment of mental health conditions (as shown in FIG. 3 and FIG. 4), subjects across groups with reported mental health conditions were analyzed and treated with personalized care, based upon sample and data analyses, which were used as model inputs to generate personalized aspects of care. Model architecture was designed to identify genetic and genomic factors, as well as gut microbiome factors associated with mental health improvement after receiving and following a personalized intervention plan, which included dietary and lifestyle interventions. Study subjects that reported mental health conditions, such as anxiety or depression, when initiating the personalized intervention plan were analyzed. Over the course of their respective personalized intervention plans, subjects were asked to rate their improvement in mental health symptoms after they had achieved at least 2% body weight loss through the digital therapeutic program. The low body weight loss threshold provided a broad range of body weight loss values in the cohort of subjects. In relation to the genomic profile, model architecture processed genetic scores for genes identified to be associated with anxiety, depression, sleep disorders (e.g., insomnia), and obesity. In relation to microbiome states, model architecture generated and processed taxonomic annotations from subject samples, summarized at the level of genera and functional pathways. Methods and results are further described in more detail below.

Subject Enrollment and Subject Characteristics: Subjects were provided with a questionnaire through an online interface, where the questionnaire included questions regarding mental health condition presence associated with anxiety, depression, and sleep disorders (e.g., insomnia, apnea, etc). Subjects who indicated a positive answer to having anxiety or depression were asked to rate the intensity of their anxiety and depression symptoms on a scale (e.g., from 0 (minimum) to 5 (maximum)). Subjects who indicated a positive answer about having a sleep disorder at baseline were asked to rate the intensity of their condition on a scale (e.g., from 0 (minimum) to 5 (maximum)). Additionally, subjects provided information on the presence or absence of symptoms associated with functional gastrointestinal disorders (FGIDs), prescribed and over-the-counter medications or supplements, alcohol intake, recreational drug usage, and demographic information, including age, gender, and height. Body mass index at baseline and follow-up was determined using the closest weight measurement to the enrollment date and date on which the survey was answered (within a window of +/-14 days). Subjects were classified as having an FGID if they reported one or more of the following six conditions: constipation, diarrhea, gassiness, bloating, heartburn/acid reflux, and abdominal pain (cramping/belly pain).

Data from subjects consuming medications (e.g., antidepressants, anti-anxiolytics, antibiotics, or antimycotics) was tagged, based upon model architecture that mapped reported medications used by subjects to the Chembl database using the database API. Model architecture further included functionality for extracting ATC codes from which identified medications prescribed for depression (e.g., N06AA, N06AB, and N06AX), anxiety (N05B) and antibiotics and antimycotics (J01B, J01C, J01D, J01E, J01F, J01G, J01M, J01R, J01X, J02, and J04) were obtained.

Personalization of intervention plans generated from model outputs was achieved by analyzing participants’ genetics, microbiome composition, lifestyle aspects, and demographics, with specific implementations of steps of methods 100, 200 described above. Based on data elements derived from these inputs, model architecture generated personalized intervention plans for each subject, which guided subjects to make incremental lifestyle changes focused on dietary regimen adjustments, with reduction of consumption (e.g., of sugar) and timing meals to optimize insulin sensitivity, reduce systemic inflammation by identifying possibly inflammatory and anti-inflammatory nutrients, and increase fiber diversity to improve gut health. Behavioral changes were implemented with the help of virtual health coaching and gamification in the application to promote habit-forming behaviors.

Sample Collection and Processing: Genome SNP Array and Microbiome Sequencing: In examples, subjects self-collected saliva samples using buccal swabs (e.g., Mawi Technologies iSwab DNA collection kit, Model no. ISWAB-DNA-1200) and gut samples (e.g., fecal samples) using swabs (e.g., Mawi Technologies iSWAB Microbiome collection kit, Model no. ISWAB-MBF-1200). Sample collection was completed by following standardized directions provided to all subjects in an instruction manual provided with a kit for sample collection. The platform then performed saliva DNA extraction, purification, and genotyping (e.g., with automated sample processing apparatus executing machine-readable instructions, as described above and below in relation to system components) using Affymetrix’s Direct to Consumer Array version 2.0 (“DTC”) on the Affymetrix GeneTitan™ platform. The platform also performed genotype calling (e.g., using Axiom Analysis Suite Software version 5.1.1). Genotypes were set to ‘missing’ if a sample from a subject did not meet the confidence threshold for making a specific call, or if the probeset did not fall into a recommended category.

The platform also performed sample processing of baseline (pre-intervention) fecal samples followed by 16S rRNA gene amplicon sequencing. The platform also performed DNA extraction (e.g., using Qiagen MagAttract Power Microbiome DNA Kit) by way of an automated liquid handling DNA extraction instrument.

Microbiome Data Analysis: The platform also amplified and sequenced the V3-V4 region of 16S rRNA gene (e.g., using the Illumina MiSeq platform) using 2 × 300 bp paired-end sequencing. The platform demultiplexed sequence reads and generated amplicon sequence variants (ASVs) using DADA2 in QIIME2 (version 2020.8). In examples, the platform trimmed primers and low quality bases (Q <30) from the reads. Computing components with architecture for transforming input data performed taxonomic annotation using a Naive Bayes classifier against the 99% non redundant Silva 138 reference database. In examples, model architecture was structured to exclude hits to mitochondria, chloroplast, eukaryota, and unassigned taxa at the phylum level. Samples for subjects who reported antibiotic consumption were excluded from the downstream analysis, according to model architecture. Furthermore, any ASV not observed more than once in at least 10% of the samples were filtered out to remove ASVs with a small mean and trivially large coefficient of variation. The abundance matrix was rarefied at even depth (n=36,000 reads per sample with 500 iterations) using the ‘q2-repeat-rarefy’ plugin from QIIME2. The abundance matrix was then agglomerated at the genus level, resulting in 178 taxa across 344 samples. Model architecture further filtered any taxa with read counts less than 10. Furthermore, the raw abundance data were subjected to centered-log ratios (CLR) transformation. Model architecture further include a microbial functional prediction component that implemented the q2-picrust2 plugin (version 2021.2) in QIIME2. Resulting abundance matrices comprising KEGG Orthologs were then used to obtain the abundance of specific pathways related to neuroactive metabolites produced by gut microbes (e.g., ‘Gut-Brain Modules’ (GBMs)). Predicted gene abundances encoding these metabolites were derived using the Omixer-RPM package (version 0.3.2).

Model architecture was further structured to identify gut network enterotypes and their relationships with mental health improvement. In a specific example, gut network enterotypes and relationships were identified using an ecological network analysis model that implemented a top-down approach to process complex microbiome interactions, where training of machine learning models identified the ecological groups correlated with mental health conditions and BMI, and the key drivers of those sub-communities. Trained models were structured to return valuable biomarkers, including new targets for synbiotics to modify community patterns, with reductions in data complexity with graph analyses. Network Module communities associated with mental health and BMI conditions, along with direction of an effect upon such conditions, are further described in more detail below, where each community has a set of microbiome features associated with it and also a subset of microbiome features that are representative of characteristics of the community, such that they can be considered the most relevant. Prebiotic or other food components known to impact those network module communities and microbiome features (e.g., by impacting key taxa drivers) were used to generate personalized intervention plan elements to improve outcomes.

Genetic Data Analysis: Model architecture unified probe level genotype calls at the variant level, and set multiallelic sites with discordant genotype calls among different probes as missing. Files (e.g., VCF files) were left normalized with bcftools version 1.14 (using htslib version 1.14). Beagle version 5.3 was used for phasing missing data imputation and phasing and imputation, where data for imputation included only SNPs and Indels, removing sites with allele counts <2 and left normalization with beftools, and converting filtered and phased VCF files to bref3 format using the bref3.28Jun21.220.jar provided by the Beagle software suite (processed 1 KG data). Model architecture included 13.478.023 variants on downstream analyses sites imputed with r2>=0.8 or chip-genotyped. All analyses were orchestrated by a SnakeMake pipeline. Model architecture merged un-imputed genotype data with the 1 KG data, including only sites with MAF > 1% and genotype missingness < 10%, and included performance of a PCA analysis and calculation of the first20 PCs using Plink2 on the combined dataset.

A selection of main five super populations defined by the 1KG data were used to estimate a sample’s ancestry composition. Training of model architecture included training of a random forest classifier (e.g., implemented on scikit-learn) with max depth = 8 and number of trees = 100 using 75% and 25% for training and test datasets, respectively, obtaining Matthew’s correlation coefficient of 0.98. Twenty-three traits were selected based on being digestive system traits comorbid with mental health disorders, anxiety, and sleep conditions, or mental health disorders that are known comorbidities of digestive system conditions focused on IBS, IBD, and obesity. Model architecture included analysis of each trait’s genetic and genome-wide association studies (GWAS), and extracted summary statistics, including chromosome, position, effect allele, effect size, and ancestry of discovery and replication populations. Genetic scores were calculated as features by multiplying the beta value or logarithm of the odds ratio by the number of risk alleles of each subject, and the mean overall genetic variants included on the panel. All genetic scores were coded to be interpreted such that a larger genetic score was associated with increasing inherited genetic predisposition to a particular condition (e.g., mental health condition).

After performing initial genetic and microbiome data processing steps described above, data generated from processed samples was prepared and processed by the platform according to the exemplary statistical model architecture aspects described as follows:

Statistical Model Architecture: To identify genetic and microbiome predictors associated with each of the outcomes, statistical model architecture was structured for univariate analyses, utilizing a multiple regression with demographic variables. For anxiety symptoms, depression symptoms, and sleep condition symptoms at baseline, logistic regression was used. To model improvements over time in intensity/severity of symptoms, Poisson regression was used, with the outcome being the intensity at T1 and offset being the intensity at T0. In all models, input demographic variables included: FGID (e.g., the self-reported status of a diagnosis of a functional gastrointestinal disorder); gender (e.g., biological gender); BMI at To; Age; weight loss (e.g., categorized as those with no change, those who lost 0 to 5%, 5-10% or more than 10% of their body weight at T1 in relation to To); and five principal components calculated using the genetic ancestry analyses described previously. Linear regression was fitted using the statsmodels python package v0.13.2 using the Binomial and Poisson families with the identity and links for logistic and Poisson regression, respectively, and with the HC₃ covariance matrix as suggested earlier. From these regression models, model architecture identified microbiome and genetic factors measured at baseline that are associated with increased prevalence of mental health conditions at baseline and different levels of improvement in mental health at follow-up (which were subsequently used to guide generation and provisional of personalized intervention plans digitally). For the logistic models (linear models with Binomial family link) a regression coefficient greater than zero was interpreted as an increasing prevalence of self-reported illness with an increasing abundance of microbiome factors or a higher value of the genetic scores. For the Poisson regression models, a regression coefficient greater than zero was interpreted as a higher abundance of microbiome factors or a higher value of genetic scores being associated with less than an average improvement in the outcome. Model architecture further applied corrections for multiple hypothesis tests using the False Discovery Rate (FDR) as implemented on the function “multiple tests” of the statsmodels python package and selected statistically significant results with an FDR ≤ 0.15.

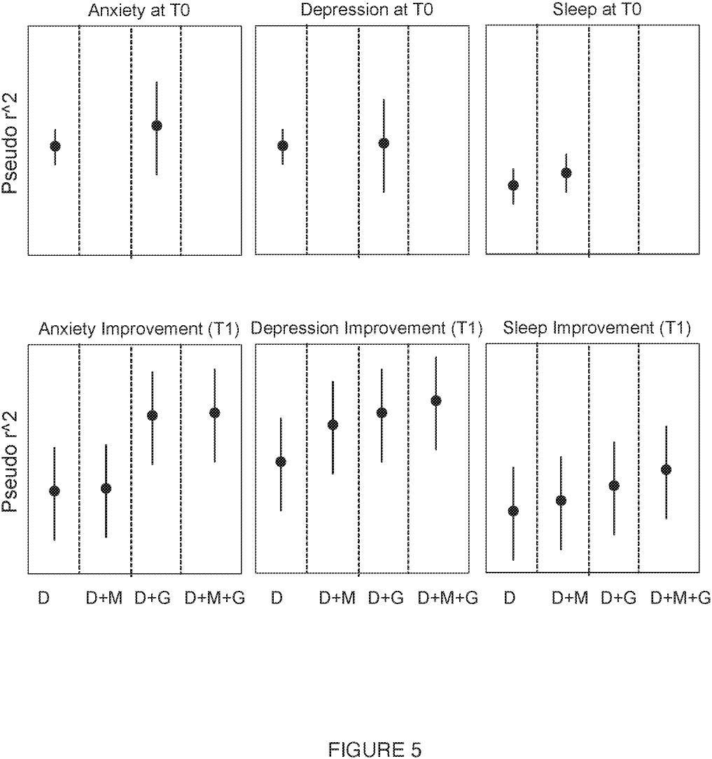

Model Comparisons. Testing, and Refinement: Variations of models were generated, and comparisons between four different models were refined and tested (results of which are shown in FIG. 5). Regression models for demographic (D), demographic and microbiome predictors (D+M), demographic and genetic predictors (D+M), and all three sets of predictors (D+M+G) were generated and evaluated for performance, in relation to predictive power and improvement in outcomes. For microbiome and genetic predictors, variables identified on univariate analyses with an FDR ≤ 0.15 were included. Model architecture included functionality for performing singular value decomposition (SVD) analysis with the microbiome variables, bacterial genera, and functional pathways, in order to avoid collinearity in the regression models. The singular vectors were as predictors on the regression models. The same number of singular vectors as the number of microbiome variables used for the SVD were selected. Adjusted pseudo r-squared values were implemented, which allow comparison between models with a different number of predictors. Models comparison was performed using Cox-Snell pseudo r-squared adjusted using Pratt’s methods to correct the different predictors in the other models. To estimate the mean, median, standard deviation, and percentiles of the pseudo r-squared values, model architecture and evaluation included performance of bootstrapped analyses with 1001 bootstrap replicates. Bootstrap values were bias-corrected by subtracting the absolute value of the difference between the mean pseudo r-squared of the 1001 bootstrap replicates and the value obtained from the original non-bootstrapped data. This ensured that the mean of the bootstrap distributions is the same as the pseudo r-squared obtained from the original non-bootstrapped data. The bootstrap analysis is not meant for hypothesis testing but to provide uncertainty estimates for the pseudo r-squared reported.

To assess the effect of potential confounders (e.g., medications and demographics) on predictions with mental health outcomes and generated outputs for personalized intervention plans, a multivariate analysis was performed using PERMANOVA with 999 bootstrap iterations based on Bray-Curtis dissimilarity with the vegan package in R. Age explained the highest variation in the microbiome at baseline, and to reduce its confounding effect, PERMANOVA models were run by controlling for age by stratifying it as blocks (strata=age). Linear regression models with CLR transformed taxa abundances as outcomes were also implemented, with inclusion of potential confounders and their interaction with mental health as predictors.

Mental Health Composite Score: Examples of the methods 100 can include generating a mental health composite score derived from various features, where the mental health composite score combines information from different genetic and/or microbiome features, each feature having a relative (non-zero) contribution to the final score. Exemplary features and weights are provided as follows (feature:weight): Shannon Diversity Index: 0.01; Acetate biosynthesis: 1.344854532348084; Butyrate biosynthesis: 1.5118856546972748; Propionate biosynthesis: 1.7232265089435275; GABA biosynthesis: 0.20770700134697656; Tryptophan synthesis: 0.9389834192791563. Exemplary features and weights can thus include a weighted aggregation of the following features: Shannon Diversity Index, Acetate biosynthesis, Butyrate biosynthesis, Propionate biosynthesis, GABA biosynthesis, and Tryptophan synthesis, with individual weights between 0.01 and 2. As such, the set of transformation operations can include generating a mental health composite score derived a weighted aggregation of a set of features comprising: a Shannon Diversity Index, an Acetate biosynthesis feature represented in the set of samples, a Butyrate biosynthesis feature represented in the set of samples, a Propionate biosynthesis feature represented in the set of samples, a GABA biosynthesis feature represented in the set of samples, and a Tryptophan synthesis feature represented in the set of samples.

The composite score can be calculated as the overall sum of the multiplication between the abundance of each microbiome feature by its weight. The composite score can alternatively be calculated as an aggregation of weights and abundances of features according to another suitable formula. Composite scores for other conditions can also be calculated. The weights were optimized using the Broyden-Fletcher-Goldfarb-Shanno algorithm and a a linear function. A composite score can be normalized in regards to a reference cohort to understand its expected value and standard deviation and particular values can be defined using the distribution alone or combined with independent information (e.g., a distribution of the score in regards to known mental health classification and Dx methods) to determine different continuous or categorical risk level assignment values, where the relative risk level assignment values can indicate risk of increasing symptom severity or risk of a critical state of a condition.

In more detail, the composite score was designed to indicate a directional shift on its value, where the directional shift is associated with improvement in symptoms. The weights were optimized in such a way that the score increases as symptoms improve, where the optimized weights provide a better discrimination of health improvement than non-optimized weights. Composite scores between groups of subjects that at T2 lost >3% body weight and those that gained weight were compared, across multiple time points. Using the optimized weights, the ratio between the composite score at a later time point (e.g., T2) to an earlier time point (e.g., T1) indicated a change in the score. The mean ratio for subjects that lost >3% weight was 1.06045665 and for those that gained weight was 1.00550161. As such, the ratio between these (lost >3% body weight / gained weight) is 1.054654352 and provides an indicator of the change of the score when subjects improve their health (lose >3% body weight). Using non-optimized weights (equal weighting of each microbiome feature) the results on the same subjects are: The mean ratio for individuals that lost >3% weight was 1.008531438 and for those that gained weight was 1.003689219. Their ratio is 1.004824421. We can see the composite score improves more associated with improvement in symptoms when using the optimized scores, where the improvement corresponds to a 4.96% better improvement.

The change in the composite score from T1 to T2 was also evaluated by calculating the difference (T2 - T1) and ratio (T2/T1) between the two time points for each subject and calculating the median for each group of subjects. The change in the score is larger for individuals that lose >3% body weight compared to individuals who gained weight, also indicating correspondence of score changes with level of improvement. The median of the difference of weight gain from T1 to T2 was 7.35, the median of the ratio of weight gain from T1 to T2 was 1.65, the median of the difference of weight loss percent >3% from T1 to T2 was 12.82, and the median of the ratio of weight loss percent >3% from T1 to T2 was 2.80.

Results: Subjects were enrolled on average 88.3 days (median = 64 and std = 67.7 days), and reported changes in symptom severity/intensity for anxiety, depression, and insomnia. Overall, study participants lost on average 5.4% body weight during the study, and more than 95% reported having an improvement in at least one mental health outcome. Analysis of the microbiome data from subjects identified 178 genera and 42 functional pathways having power in affecting outcomes. Analysis of the genetic data identified 23 genetic scores having power in affecting outcomes.

Cohort demographic characteristics:_The mean BMI of the subjects was 34.6, corresponding to obese class 1 individuals. At the time of answering the follow-up questionnaire, most subjects lost between 5 to 10% body weight. This cohort had a high prevalence of subjects with FGID (84%) and females (79%). 284 (86%) of the subjects reported taking antidepressants (40 or 12%) or anti-anxiolytics (244 or 74%) at baseline. Genetic ancestry analyses identified the majority of individuals as of European ancestry (43%), followed by African (28%), Americans (20%), east Asians (7%), and southeast Asians (1%). Results include coverage of first five principal components from the genetic ancestry analysis as covariates in all analyses.

Mental Health – Anxiety: For the exemplary group of subjects, after participating in personalized intervention programs generated by the invention(s) described, 59% of subjects reported improvements in anxiety symptoms, with 22%, 24%, and 13% reported improving in 1, 2 and 3 scale points. Three genetic scores, irritable bowel syndrome (IBS), body mass index (BMI), and obstructive sleep apnea (OSA), reached statistical significance for association with changes in anxiety and generation of outcomes with personalized intervention. IBS correlated with small improvement and BMI and OSA with a large improvement in anxiety intensity scores after intervention. Seven bacterial genera were statistically associated with changes in anxiety and generation of outcomes with personalized intervention. The abundances of four taxa, Dorea, Ruminococcaceae_UBA1819, Oscillospiraceae_UCG003, and Eubacterium ventriosum group, correlated with a small improvement, and three, Ruminococcaceae_DTU089, Prevotella, and Adlercreutzia, with a large improvement in anxiety scores. An increasing abundance of genes of the bacterial functional pathway kynurenine synthesis (MGB004) was associated with a small improvement in anxiety symptoms at follow-up.

Mental Health – Depression: For the exemplary group of subjects, after participating in personalized intervention programs generated by the invention(s) described, 51% of subjects reported improving their symptoms with 21%, 21%, and 9% reported improving in 1, 2, and 3 scale points. Two genetic scores were directly associated with a large decrease in intensity scores, OSA and AUD, and height was associated with a small improvement. Nine bacterial genera and four functional pathways were associated with improvement in depression intensity and generation of outcomes with personalized intervention, eight of which were associated with small improvement and five with large improvement

Mental Health – Sleep: For the exemplary group of subjects, after participating in personalized intervention programs generated by the invention(s) described, 66% of subjects reported improving their symptoms with 29%, 18%, and 19% reported improvement in 1, 2, and 3 scale points. Two genetic scores were statistically significant, with one associated with a large decrease in intensity scores, type 1 diabetes (T1D), and one associated with a small decrease in intensity scores, type 2 diabetes (T2D). Two bacterial genera were associated with improvement in insomnia intensity, Butyricimonas associated with a small improvement, and Roseburia with a large improvement. Finally, one functional pathway was associated with a large improvement in insomnia, nitric oxide synthesis II (nitrite reductase).

Mental Health – General: Logistic regression models were implemented by model architecture to evaluate the association between genetic scores and microbiome components and other data inputs with depression, anxiety, and sleep problems at T0 (baseline) and subsequent time points. Two genetic scores, namely alcohol use disorder (AUD) and major depressive disorder (MDD), were statistically associated with an increased prevalence of depression and anxiety. Two microbial functional pathways were associated with sleep problems at baseline, Menaquinone synthesis (vitamin K2) I (MGB040) with increased prevalence of sleep problems at T0, and inositol degradation (MGB038) with decreased prevalence of sleep problems at T0.

Generally, genetic scores used for predictions and personalized intervention plans were derived from SNP features, where a set of SNP features can include: characterizations of risk alleles detected for the subject, the risk alleles individually or in combination providing a measure of inherited risk (e.g., such that the method includes determining inherited risk) for at least one of: irritable bowel syndrome, obstructive sleep apnea, alcohol use disorder, major depressive disorder, and type I diabetes. SNP features can incldue characterizations of risk alleles detected for the subject, including risk alleles associated with Genome Wide Association Studies (GWAS) corresponding to: risk allele variants for irritable bowel syndrome from GWAS study GCST90016564, risk allele variants for obstructive sleep apnea from GWAS study GCST011921, risk allele variants for alcohol use disorder from GWAS study GCST012354, risk allele variants for major depressive disorder from GWAS study GCST007342, and risk allele variants for type I diabetes from GWAS study GCST90013445.

Generally, microbiome features used for predictions and personalized intervention plans included: features associated with Dorea genus, Ruminococcaceae genus UBA1819, Oscillospiraceae genus UCG003, Eubacterium ventriosum group, Ruminococcaceae genus DTU089, Prevotella, and Adlercreutzia in relation to anxiety symptoms of the set of mental health condition symptoms; features associated with Clostridium innocuum group, Oscillospiraceae genus UCG003, Anaerostipes, Eubacterium ventriosum group, Lactobacillus, Negativibacillus, Prevotella, Oscillibacter, and Actinomyces in relation to depression symptoms of the set of mental health condition symptoms; and features associated with Butyricimonas and Roseburia in relation to sleep symptoms of the set of mental health condition symptoms.

Additionally or alternatively, the set of microbiome features comprises features associated with Dorea genus, Ruminococcaceae genus UBA1819, Oscillospiraceae genus UCG003, Eubacterium ventriosum group, Ruminococcaceae genus DTU089, Prevotella, Adlercreutzia and Kynurenine synthesis in relation to anxiety symptoms of the set of mental health condition symptoms; features associated with Clostridium innocuum group, Oscillospiraceae genus UCG003, Anaerostipes, Eubacterium ventriosum group, Lactobacillus, Negativibacillus, Prevotella, Oscillibacter, Actinomyces, MGB053: Butyrate synthesis II, p-Cresol synthesis, Nitric oxide synthesis II (nitrite reductase) and Nitric oxide degradation I (NO dioxygenase) in relation to depression symptoms of the set of mental health condition symptoms; features associated with Butyricimonas, Roseburia, Menaquinone synthesis (vitamin K2) I, Inositol degradation, and Nitric oxide synthesis II (nitrite reductase) in relation to sleep symptoms of the set of mental health condition symptoms; network community GMNC-1 comprised of a set of taxa including Flavonifractor, Clostridium innocuum group, Ruminococcaceae UBA1819, Eggerthella, Oscillibacter, Hungatella, Eisenbergiella, Anaerotruncus, Holdemania, Erysipelatoclostridium, Eubacterium nodatum group, Colidextribacter, Dielma, Bilophila, Eubacterium fissicatena group, Gordonibacter, Parabacteroides, Ruminococcus torques group, Clostridium methylpentosum group, Lachnospiraceae FCS020 group, with its set of keystone taxa defined by intra-community connectivity being Flavonifractor, Clostridium innocuum group, Ruminococcaceae UBA1819, Eggerthella, Oscillibacter and Hungatella, and with its first eigenvector of the spectral decomposition of the community members among the samples studied being positively associated with improvement on depression and anxiety; GMNC-4 comprised of a set of taxa including Faecalibacterium, Lachnospiraceae NK4A136 group, Monoglobus, Lachnospiraceae ND3007 group, Eubacterium xylanophilum group, Oscillospiraceae UCG-003, Anaerostipes, Fusicatenibacter, Parasutterella, Anaerovoracaceae Family XIII UCG-001, Eubacterium hallii group, Escherichia-Shigella, Eubacterium eligens group, Actinomyces, with its set of keystone taxa defined by intra-community connectivity being Faecalibacterium, Lachnospiraceae NK4A136 group and Monoglobus, and with its first eigenvector of the spectral decomposition of the community members among the samples studied being positively associated with depression and anxiety risk or propensity and positively associated with improvement on depression and anxiety; and GMNC-3 comprised of a set of taxa including Unannotated Oscillospiraceae (Family), Lachnospiraceae UCG-010, Anaerovoracaceae Family XIII AD3011 group, Unannotated Lachnospiraceae (Family), Incertae Sedis, Butyricicoccus, Paludicola, Lachnospiraceae UCG-001, Roseburia, Lachnospira, Caproiciproducens, Negativibacillus, Adlercreutzia, Eubacterium ventriosum group, Eubacterium brachy group, Ruminococcaceae DTU089, Akkermansia, Lachnospiraceae CAG-56, Erysipelotrichaceae UCG-003, with its set of keystone taxa defined by intra-community connectivity being Anaerovoracaceae Family XIII AD3011 group, Unannotated Lachnospiraceae (Family), Incertae Sedis, Butyricicoccus, Paludicola, Lachnospiraceae UCG-001, Roseburia, and with its first eigenvector of the spectral decomposition of the community members among the samples studied being negatively associated with BMI and positively associated with improvement in depression.

This study identified 8 genetic scores, 15 microbiome genera, and 7 functional pathways associated with improvement in anxiety, depression, insomnia, or anxiety/depression and sleep problems at baseline. Trained models identified associations between a higher abundance of Kynurenine synthesis (MGB004) and improvement in anxiety intensity. Kynurenine is a catabolic product of the tryptophan-Kynurenine metabolism and is further metabolized into kynurenic acid or quinolinic acid. Trained models identified associations between the gut microbial pathway involved in p-Cresol synthesis (MGB015) and improvement in depression intensity. Interestingly, trained models identified associations nitric oxide synthesis II (MGB026: nitrite reductase) and a large improvement in depression and insomnia intensity. In contrast, the nitric oxide degradation I pathway (MGB027: nitric oxide dioxygenase) was associated with a large improvement in depression intensity only. Increased NO degradation by the gut microbiome may mimic the effects of pharmacological treatments.

The association between the increasing abundance of butyrate synthesis II (MGB053) and a small improvement in depression is in the opposite direction of the generally reported relationship between short-chain fatty acids and mental health. The associations generated by the trained models may reflect a different pattern in regards to the nature of the personalized interventions. This could be explained because the intervention increases dietary fiber, which is known to increase the relative abundance of butyrate-producing microbes and those producing other SCFA.

Additional associations identified using trained models, for generating personalized interventions: Associations between depression and Oscillospiraceae_UCG003), Eubacterium ventriosum group, Lactobacillus, Prevotella, and anxiety with Ruminococcaceae_UBA1819 and Ruminococcaceae_DTU089. Butyricimonas and Roseburia were associated with improvement in insomnia.

Interestingly, several genera were systematically associated with anxiety or depression at baseline and with improvement in multiple outcomes. For instance, the Eubacterium ventriosum group was significantly associated with improvement in anxiety and depression with the same direction of effect (beta = 0.24 and 0.20). Likewise, Prevotella was associated with improvement in anxiety and depression with the same direction of effect (beta = -0.13 and -0.12), and nominally associated with anxiety or depression at baseline (beta = -0.3 and p-value = 0.0028). Oscillospiraceae_UCG003 was associated with improvement on depression (beta = 0.28) and reached a nominal association with improvement on anxiety (beta = 0.42 and p-value = 0.0048).

The association between the IBS genetic score and a small improvement in anxiety suggests that individuals at higher inherited risk for IBS improve their anxiety symptoms after our digital therapeutics intervention. Our results can be interpreted as evidence that this shared etiology have implications for therapeutic response. The association between BMI and OSA genetic scores with a large improvement in anxiety may be explained based on the direct relationship between obesity and the occurrence of OSA, and the fact that sleep disturbances and poor sleep are partially caused by OSA and OSA is associated with higher anxiety symptoms. Therefore, weight loss can lead to OSA and anxiety.

Improvement in depression was associated with the AUD genetic score, with a higher genetic score implying a more significant improvement in self-reported depression. Genetic scores for alcohol use are positively correlated with the amount of alcohol consumed. Avoiding alcohol consumption is strongly recommended as part of the digital therapeutics intervention, and removing alcohol from the diet would lead to improvement in mental health.

In addition to their co-occurrence there exists evidence of a genetic and a causal link between T2D and insomnia as supported by 2 out of 3 genetic scores evaluated with the only discordance occurring on an automatically calculated genetic score. The metabolic nature of T2D, compared with T1D, is more prone to improvement under the implemented dietary intervention, which is focused, among other objectives, to reduce insulin resistance and diabetes severity and risk, which could explain why subjects with higher genetic risk for T2D may improve less than average on their insomnia.

Analysis of subjects’ reported anxiety or depression at baseline was associated with the genetic scores of two psychiatric disorders, namely alcohol use disorder (AUD) and major depressive disorder (MDD), with a higher risk of self-reporting anxiety or depression with increasing genetic score values. Trained models identified the association between gut microbiome Menaquinone synthesis (vitamin K2) I (MGB040) and a higher prevalence of sleep problems at baseline. Trained models also identified a significant association of gut microbiome inositol degradation (MGB038) with a lower prevalence of sleep-related issues at baseline.

Multi-omics models were shown to be better correlated with mental health improvement than demographics models alone. In more detail, the ability of models combining demographic (D), genetic (G), and microbiome (M) information to explain the study outcomes: anxiety, depression, and/or sleep issues at baseline, and improvement of anxiety, depression, and sleep issues from T0 to subsequent time points was evaluated. D+M or D+G models were found to explain more of the variation of the outcomes than the D model. Additionally, the D+G+M models were always better than the D models, and at least of a similar magnitude as the best D+M or D+G model.

Sensitivity analyses performed evaluated the potential confounder effect of medication, alcohol intake, and recreational drug use on the microbiome associations identified. A PERMANOVA testing the impact of the interaction between medicines and depression or anxiety at T0 or medication and sleep problems at T0 on all bacterial genera and gut-brain modules/ functions was performed. There was no evidence of a confounding effect of anxiolytic or antidepressant drugs on either anxiety or depression or sleep problems at T0. Similarly, there was no identified confounding effect of medications when the analysis was repeated with a subset of microbial markers (both bacterial genera and functions) that were found to be significantly associated with the mental health status at baseline and change in intensity of a particular outcome at T0. Lastly, the same analyses were performed for each genus and pathway separately. There was no identified confounding effect of medication, alcohol intake, or recreational drug use on the bacterial genera and pathways identified as significantly associated with the outcomes. Medication and recreational drug use were therefore not found to confound microbiome associations with mental health. Demographic factors such as age (controlled for age by stratifying it as blocks in all PERMANOVA models), gender, and BMI were also not found to have a confounding effect of these factors on the associations of the microbiome with mental health.

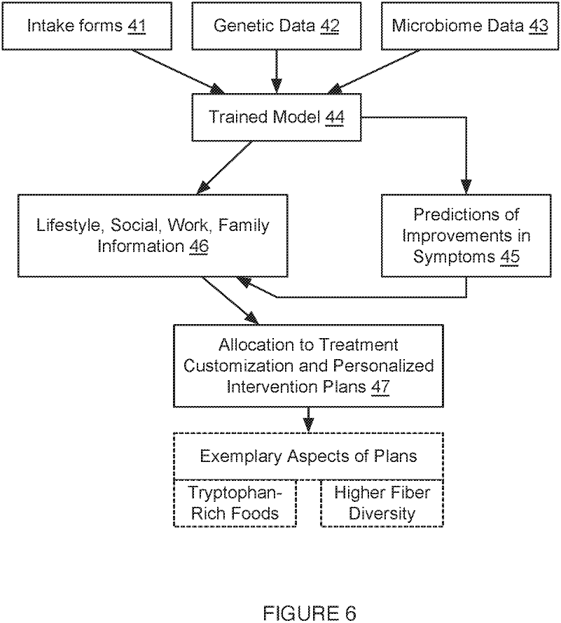

A diagram of an exemplary stratification model is shown in FIG. 6, where a trained model 44 processes data from subject intake forms 41, genetic data 42, and microbiome data 43 (as described), in order to return predicted improvements in symptoms of mental health conditions 45, with a feedback loop, integrating iterative processing of lifestyle-related, social-related, work-related, and family-related determinants of health 46. Model outputs were used to allocate food types, frequencies of consumption, lifestyle changes, pharmacological support, and/or psychotherapy-based support (e.g., with cognitive behavioral therapy, psychotherapy, etc.) in personalized intervention plans 47. Exemplary food recommendations include tryptophan-rich foods, higher fiber diversity foods, and/or other foods.

In relation to mental health conditions, an exemplary personalized intervention plan included a dietary plan configured to adjust taxonomic abundances and microbiome function represented by microorganism taxa of the baseline microbiome state, wherein the dietary plan comprises a recommended food list comprising superfood items, items to consume in moderation, items to avoid consuming, items associated with conditions comorbid with a set of mental health condition symptoms, and items associated with insulin resistance. In examples, the personalized intervention plan included provision of a prebiotic blend including ingredients rich in at least one of: inulin, flavonoids, withanolides, sitoindosides or acylsterylglucosides according to a regimen of consumption.

In examples, the methods can further include determining a response to the personalized intervention plan by the subject, based upon identification of modulation of microbial taxa associated with a combination of a) a first subset of taxa that decrease in abundance longitudinally in time and comprising: Parabacteroides, Desulfovibrio, Oscillospiraceae UCG-002, Sutterella, Akkermansia, DTU014, Anaerotruncus, Unannotated Erysipelotrichaceae Family, Clostridia vadinBB60 group, Eubacterium fissicatena group, Enterorhabdus, Christensenellaceae R 7 group, Clostridium sensu stricto 1, Oscillospirales UCG-010, Megasphaera, Unannotated Rhodospirillales Order, Unannotated Erysipelatoclostridiaceae Family, Coprobacter, Rothia, Cloacibacillus, Enterobacter that increase longitudinally, and Peptostreptococcus, Fenollaria, Paludicola, Holdemanella, Erysipelatoclostridiaceae UCG004, Agathobacter, Eubacterium ruminantium group, Enterococcus, RF39, Anaerofustis, Lachnoclostridium, Eggerthella, Phascolarctobacterium, Roseburia, Solobacterium; b) a second subset of taxa that decrease in abundance longitudinally in time and comprising gut microbial communities associated with GMNC-1, GMNC-2 and GMNC-4; and a third subset of taxa that increase in abundance longitudinally in time and comprising gut microbial communities associated with GMNC-3.

### 2.5.2 Method Exemplary Execution and Performance - Weight-Related Conditions and Longitudinal Care

In an exemplary application of the platform for prevention and/or treatment of subjects for whom weight was an issue, the platform provided components for receiving and processing samples, as well as for generating and executing personalized intervention plans in order to reduce weight of subjects according to their respective goals, an in a healthy manner.

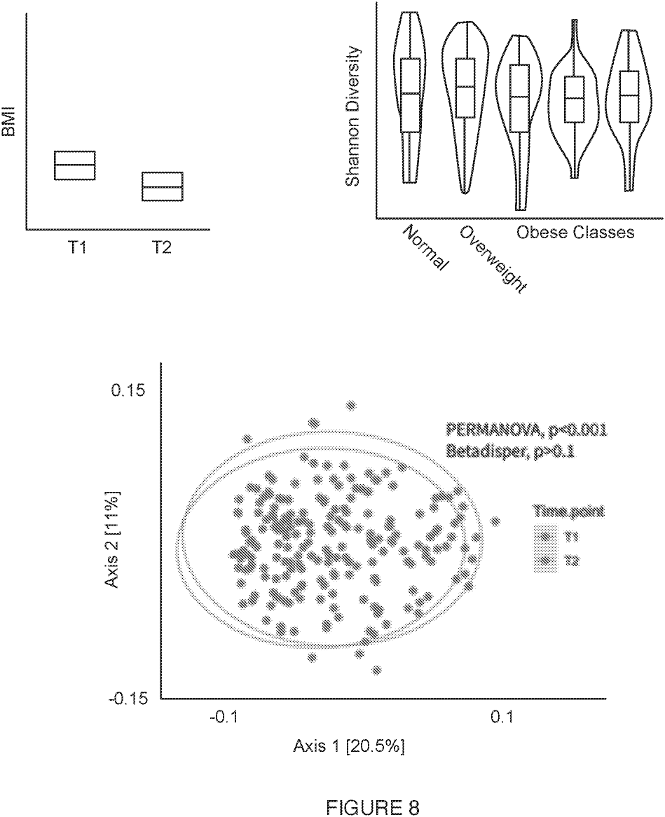

In the exemplary application, a set of 103 individuals was analyzed from their enrollment (T0) to generate, provide, and study the effects of a personalized digital therapeutic obesity management and weight loss program on their BMI and gut microbiome (measured at T1 and T2). Overall the inventions achieved a significant weight loss across the study participants with a mean weight decrease of 2.6 and 1.4 BMI units lost from T0 to T2 and T1 to T2, respectively, corresponding to a mean 7.4% and 4.9% body weight loss. Interestingly, the trained models used in the exemplary application achieved identification of significant and beneficial associations between gut microbiome diversity and weight loss. On individual association analyses, the model identified gut microbiome biomarkers associated with weight loss, for use in personalized intervention plans, for the intervention period (T1 vs T2), and involving bacterial genera, bacterial functional pathways, and bacterial community networks. Some exemplary data is shown in FIG. 8, and in Applications incorporated by reference.

Subject Characteristics and Overview: Subjects included 75.7% of females with an average age of 53.55 (Median 55.0; IQR: 44.5, 63.0), where a high percentage of subjects were suffering from FGIDs (e.g., at least one self-reported functional gastrointestinal disorder: IBS, gassiness, bloating, constipation, diarrhea or dyspepsia), while ~86% had other comorbidities along with overweight or obese conditions at baseline (To). Additionally, 35% of subjects were on prescribed antidepressants or anxiolytics, while 14.6% were using recreational drugs at baseline (T0), during enrollment. An embodiment of the model produced statistically significant weight losses in subjects, from baseline to T2, with around 80% of subjects losing weight. The average reduction in BMI from T0 to T2 across the group of subjects was 2.57 BMI units (Median: 2.6; IQR 2.1, 2.6, p-value<0.001). There was a significant reduction (p-value<0.001, Wilcoxon Signed-rank test) in weight from T1 to T2 with an average reduction of 1.6 BMI units (Median 1.4 (IQR: 0.3, 2.7). Around 14.6% of subjects lost 3-5%, 34% lost 5-10% and 28.2% lost more than 10% of body weight from baseline T0 to T2. Furthermore, 17.5%of individuals lost 3-5%, 31.1% lost 5-10% and 14.6% lost more than 10% of body weight from T1 to T2.

The embodiment of the model further included architecture for returning multivariate associations that showed that sampling time point (T1 vs. T2 PERMANOVA, p-value <0.001), Age (PERMANOVA, p-value <0.01) and Gender (PERMANOVA, p-value <0.01) were among the top drivers of overall gut microbial diversity. Interestingly, the inter-subject variation when compared between the genders (Male vs. Female) was significantly different (Betadipser test p-value = 0.01) and reduced at T2. Additionally, the consumption of prescribed antidepressants and anxiolytics and the consumption of alcohol at baseline (To) was seen to contribute significantly to the microbiome variation. BMI had a significant association with the overall diversity (PERMANOVA, p-value = 0.04) and was not confounded by other variables including age, gender, and time point, as evidenced by the interactions.

The embodiment of the model returned microbiome features and genetic features associated with BMI statuses in subjects, and were used to generate personalized intervention plans for reducing BMI and severity of other symptoms of comorbid conditions simultaneously.

Sample Collection and Processing: Subjects self-collected fecal samples using fecal swabs (Mawi Technologies iSWAB Microbiome collection kit, Model no. ISWAB-MBF-1200). Sample collection was completed by following standardized directions provided to all subjects in an instruction manual provided with a kit for sampling. Sample processing of baseline (pre-intervention) fecal samples was followed by the 16S rRNA gene amplicon sequencing. DNA extraction was performed using Qiagen MagAttract Power Microbiome DNA Kit on an automated liquid handling DNA extraction instrument. The bacterial 16S rRNA gene V3-V4 region was amplified and sequenced on the Illumina MiSeq platform using 2 × 300 bp paired-end sequencing and sequence reads were demultiplexed, denoised and ASVs generated using DADA2 in QIIME2 version 2021.4.

Microbiome Data Analysis: In total, methods included collection of 206 stool swab samples across multiple time points. Initial quality control steps included removal of primers and low quality bases, removal of ASVs classified as non bacterial sequences (such as Euryarchaeota, Chloroplast, Mitochondria) or unassigned taxa at the phylum level. Taxa were agglomerated at genus levels and those with low abundance (taxa with <10 reads in at least 10% of samples) were excluded, resulting in reduction of sparsity of the abundance matrix from 99.75% to 37.6% (with an average of 98.3% of read retention) and removal of singletons. The abundance matrix was rarefied at even depth (n=61,000 reads per sample (minimum reads across the samples) with 500 iterations) using QIIME2 (Xia 2021), resulting in 155 taxa across 206 samples. The abundance of microbial functional pathways related to gut and neuroactive metabolites was calculated with the q2-picrust2 plugin (v2.4.2) in QIIME2 (and the Omixer-RPM package (version 0.3.2). All raw abundances were centered-log ratio (CLR) transformed, unless otherwise specified.

Network Module Model Architecture: Model architecture included portions for returning network analyses of the gut microbiome profiles in order to identify 1) network modules associated with BMI and BMI changes between the time points studied, and 2) the key driving taxa of these network modules. Network modules are a set of taxa with higher levels of correlation on their abundance among them compared to other taxa. Model architecture utilized the WGCNA software package for R. Methods included preprocessing the abundance profiles by calculating the CLR transformation and setting taxa with 0 counts as NA. Model architecture used the a soft pick threshold function to explore the relationship between the power parameter and the connectivity and selected power = 1 for the analyses. Higher values led to a drastic reduction of average connectivity which strongly influenced the detection of modules which translated into detecting none or one with values of the power parameter >3. The per taxa missingness values filtered out 52 taxa with excess of missing values and for which pairwise correlations with other taxa could not be calculated. Likewise, 1 sample was removed from the analysis due to 50% missing values. Model architecture used correlation characterizations appropriate to the non-normal distribution of microbiome abundance values. Model architecture compared the results obtained using the a correlation with a signed or unsigned TOMType and we found the resulting modules were identical. The output of the “blockwiseModules” function provides an assignment of each taxa to a network module, and a summary of the abundance patterns of the modules and calculates the first eigenvector which summarizes the main trend of the abundance matrix of the taxa associated with each module. Model architecture calculated the correlation between each taxa CLR value and the module eigenvector to identify the taxa that are key drivers of the network module. Exemplary module characteristics, eigenvalue relationships with BMI, and cluster dendrograms are provided in Applications incorporated by reference.

Statistical Model Architecture: Model architecture implemented PERMANOVA, which performs community-level multivariate association with variables, based on the abundance matrix using the Bray-Curtis dissimilarity, using the vegan package (adonis2 function, strata= user.id) in R. Further to test for homogeneity of multivariate dispersions (comparing inter-individual variations) between groups, model architecture used betadisper test (i.e., a multivariate analogue of Levene’s test for homogeneity of variances). Comparisons of the diversity indices between time points (T1 vs. T2) were tested statistically using Wilcoxon-signed rank test with FDR corrections.

In order to identify taxa and functional pathways associated with BMI and/or changes between T1 to T2, model architecture implemented linear mixed-models as implemented on the GAMLSS software package for R. In particular, model architecture used a regression formula. Model architecture performed multiple corrections using the local FDR methodology.

Model architecture tested the association between a network module’s eigenvector and BMI and time point using linear mixed-models. Model architecture identified modules that were associated with either BMI or time-point and those that are associated with both. Results were corrected for multi-testing using Sidak’s methodology which calculates the family-wise corrected p-values p^Sidak =1-(1-p)^k where p is the univariate p-value and k = 5 is the number of hypotheses tested.

Microbiome Features (taxonomic features, pathways, and networks): Model sub-architecture (e.g., implementing a PERMANOVA model) generated results indicating that a time point during the intervention (Early intervention phase (T1) vs. later in the intervention (T2)) was contributing to the variation in microbial diversity, indicating a shift in gut microbial diversity structure in individuals participating in the dietary intervention, and was independent of the variation explained by BMI. The embodiment of the model applied a Generalized Additive Model for Location, Scale, and Shape (GAMLSS) with a zero-inflated beta (BEZI) family (GAMISS-BEZI) regression model to analyze the association of variables with individual microbial taxa (at genus level, at other levels) and predicted abundances of pathways of interest. The embodiment of the model returned 62 genera showing significant associations with BMI in subjects (and were used to generate personalized intervention plans) after correcting for multiple testing (FDR < 0.05). Of note, Megasphaera, Cloacibacillus, and members of the Lachnocpiraceae family (including Lachnoclostridium, unannotated genera such as Lachnospiraceae UCG-001 and Lachnospiraceae CAG-56) were associated with increasing BMI. Hydrogenoanaerobacterium, Solobacterium, Desulfovibrio, Phascolarctobacterium, Christensenellaceae R7 group, and unannotated genera from Oscillospiraceae family (including UCG_002, UCG_005, NK4A214_group) were associated with lower BMI. In total, 36 genera were seen to change significantly at time point T2 (FDR < 0.05), indicating changes associated with the intervention. Of note, Akkermansia, Anaerotruncus, Coprobacter, and Rothia increased substantially at time point T2.

The embodiment of the model further evaluated 18 taxa that were associated with both BMI and time point. Unexpectedly, 9 out of these 18 taxa had a pattern of association indicating that the intervention improved their abundance (i.e., they were negatively associated with BMI and increased at T2, or were positively associated with BMI and decreased at T2). In particular, those associated with lower BMI, (i.e., Clostridia vadinBB60 group, Christensenellaceae R7 group, Enterobacter, Desulfovibrio, and Oscillospiraceae UCG-002) were seen to be enriched at time point T2, and genera associated with higher BMI (i.e., Agathobacter, Fenollaria, Lachnoclostrdium and Roseburia) depleted substantially at T2. Model outputs returned a few taxa that increased at T2 and were associated with higher BMI (i.e., Cloacibacillus, Enterohabdus, Megasphaera, and Sutterella, while those that were associated with lower BMI and decreased at T2 such as Eggerthella, Erysipelatoclostridiaceae UCG-004, Eubacterium ruminantium group, Phascolarctobacterium and Solobacterium).

Additionally, the embodiment of the model processed input data to identify microbial functions, and returned 21 pathway modules that were significantly associated with BMI (FDR < 0.05) modulation (and used to generate personalized interventions for subjects). Pathways associated with the degradation of simple sugars (carbohydrates) such as arabinose (MF0014), sucrose (MF0010) and melibiose (MF0009) were significantly associated with higher BMI. Biosynthesis of propionate (MGB054, MGB055, and MF0094) a Short Chain Fatty Acid (SCFA), GABA (γ-Aminobutyric acid) synthesis (MGB021), putrescine degradation (MF0082), degradation of amino acids such as lysine (MF0057), serine (MF0048) and phenylalanine (MF0024), and triacylglycerol degradation (MF0064) were negatively associated with higher BMI. The association between time point and predicted functions that reached significance after multiple testing correction (FDR < 0.05) included pathways associated with nitric oxide synthesis, degradation, and metabolism (MGB026, MGB027, MGB028), inositol degradation (MGB038), GABA degradation (MGB019), and pyruvate dehydrogenase complex (MF0072), which were seen at higher abundance at T2. However, the pathway associated with histamine synthesis (MGB009) was seen depleted at T2. In line with the results for bacterial taxa, the embodiment of the model returned outputs indicating a functional pathway, histamine synthesis, which was associated with both BMI and time point.

Weighted network analyses architecture of the model identified 5 network modules representing an overall organizational structure of the gut microbiome of the samples acquired. These network modules ranged in size between 13 to 20 taxa. A module’s eigenvector was used as a proxy for the abundance of the taxa of each module and was tested for statistical associations with BMI and the change between T1 to T2. Module 2 was associated with BMI with a positive correlation (Sidak p-value = 0.037). Module 3 was associated with BMI with a negative correlation (Sidak p-value = 0.015) and also showed a change in the abundance pattern between T1 and T2 with a negative correlation (Sidak p-value = 0.024). For each of these modules, the embodiment of the model identified the taxa driving the co-abundance pattern captured by the module eigenvector. Several of the genera identified on univariate analyses are part of these network modules and are among those identified as key taxa within the modules. Taxa associated with module 2 include: Anaerovoracaceae_Family_XIII_AD3011_group, Unannotated Oscillospiraceae (Family), Roseburia, Incertae_Sedis, Lachnospiraceae_UCG-001, Butyricicoccus, Lachnospira, Akkermansia, Lachnospiraceae_UCG-010, Eubacterium_ventriosum_group, Negativibacillus, Unannotated Lachnospiraceae (Family), Paludicola, Adlercreutzia, Caproiciproducens, Erysipelotrichaceae_UCG-003, Eubacterium_brachy_group, Lachnospiraceae_CAG-56, and Ruminococcaceae_DTU089. Taxa associated with module 3 include: Oscillospiraceae_UCG-005, Christensenellaceae_R-7_group, Clostridia_vadinBB60_group, Unannotated Ruminococcaceae (Family), Oscillospiraceae_UCG-002, Oscillospiraceae_NK4A214_group, Alistipes, Oscillospirales_UCG-010, Unannotated Christensenellaceae (Family), Lachnospiraceae_UCG-004, Defluviitaleaceae_UCG-011, Odoribacter, Dorea, Butyricimonas, and Sutterella.

The weighted network analyses identified five network modules as the overarching structure of the gut microbiome among these individuals. Module 2 was significantly associated with BMI with a negative correlation and several of the genera on the module were with BMI with the same directions of effect, including Akkermansia, Roseburia, Butyricicoccus, Lachnospira, and Eubacterium ventriosum group. Among these, Roseburia and Akkermansia were significantly associated with BMI and changed their abundance between T1 and T2. Module 3 was significantly associated with BMI with a positive correlation and also showed a significant decrease in abundance at time point T2. In line with model-returned indications for module 2, several of the genera included on module 3 were associated with BMI and individually were associated with BMI and changed their abundance between T1 and T2, including Oscillospiraceae, Christensenellaceae, Alistipes, and Sutterella. These findings support the notion that gut microbiome changes associated with obesity, BMI and dietary changes do not occur on individual taxa on isolation but on microbial communities whose members are dependent and complementary to each other, with respect to nutrient utilization, metabolite production and niche occupation.

Digital Therapeutics Personalized Intervention Plans: Personalization of dietary plans was achieved by analyzing participants’ genetics, gut microbiome, lifestyle, and demographics, from returned model outputs described above. Based on these data, the personalized programs encouraged participants to make incremental lifestyle changes focused on reducing sugar consumption and timing meals to optimize insulin sensitivity, reduce systemic inflammation by identifying possibly inflammatory and anti-inflammatory nutrients, and increase fiber diversity to improve gut health, accounting for their specific genetics and microbiome compositions, with respect to functional pathways and network features. Behavioral changes were implemented with the help of virtual health coaching and a mobile device application.

In an example, for the reduction in BMI, the personalized intervention plan was generated based upon detection of a set of microbiome features from the baseline microbiome state, associated with: Christensenella genera and Oscillospiraceae family; and functions comprising: mucin degradation anti-inflammatory Short-Chain Fatty Acids (SCFAs) synthesis, and propionate synthesis pathways. Additionally or alternatively, for the reduction in BMI, the personalized intervention plan was generated based upon detection of genes associated with simple sugar metabolism, histamine synthesis, and nitric oxide degradation pathways from the SNP genomic profile.

In another example, for the reduction in BMI, the personalized intervention plan is generated based upon detection of a set of microbiome taxa from the baseline microbiome state, associated with a) a first subset of taxa that have a negative association with BMI and comprising: Solobacterium, Eubacterium ruminantium group, Erysipelatoclostridiaceae UCG 004, Catenibacterium, Desulfovibrio, Enterobacter, Eubacterium nodatum group, Paraprevotella, Desulfovibrionaceae Family, Hydrogenoanaerobacterium, Coriobacteriales Incertae Sedis Family, Oscillospiraceae UCG 002, Eubacterium siraeum group, Christensenellaceae R 7 group, Phascolarctobacterium, Clostridia vadinBB60 group, Ruminococcaceae Family, Barnesiella, Oscillospiraceae UCG 005, Caproiciproducens, Ruminococcaceae UBA1819, Ruminiclostridium, Erysipelatoclostridium, Oscillospiraceae Family, Christensenellaceae Family, Candidatus Soleaferrea, Alistipes, Marvinbryantia, Lachnospiraceae NK4A136 group, Oscillospiraceae NK4A214 group

Anaerovoracaceae Family XIII AD3011 group, Eggerthella, Anaerofilum, Gordonibacter, and b) a second subset of taxa that have a positive association with BMI and comprising: Actinomyces, Lachnoclostridium, Granulicatella, Fusicatenibacter, Lachnospiraceae Family, Streptococcus, Lachnospiraceae CAG 56, Lachnospiraceae UCG 001, Sutterella, Bifidobacterium, Oscillospiraceae UCG 003, Dialister, Prevotella, Eggerthellaceae Family, Candidatus Stoquefichus, Sellimonas, Ruminococcus gauvreauii group, Roseburia, Oscillospira, Enterorhabdus, Muribaculaceae, Allisonella, Agathobacter, Peptococcus, Acidaminococcus, Fenollaria, Megasphaera, and Cloacibacillus.

Additionally or alternatively, in another example, for the reduction in BMI, the personalized intervention plan is generated based upon detection of microbial genes associated with a) a first subset of pathways that have a negative association with BMI and comprising: putrescine degradation, GABA synthesis, phenylalanine degradation, Histamine synthesis, triacylglycerol degradation, lysine degradation, Propionate synthesis, lactate consumption, mucin degradation and serine degradation; and b) a second subset of pathways that have a positive association with BMI and comprising: sucrose degradation, fructan degradation, melibiose degradation, arabinose degradation, hydrogen metabolism, arabinoxylan degradation, simple sugar metabolism (such as arabinose, sucrose and melibiose), and cysteine biosynthesis/homocysteine degradation.

Additionally or alternatively, in another example, for the reduction in BMI, the personalized intervention plan is generated based upon detection of microbial genes associated with a set of microbiome communities, comprising a) a first subset of communities having a first eigenvector of a spectral decomposition of community members of the set of microbiome communities negatively associated with BMI and comprising: community GMNC-3; and b) a second subset of communities having the first eigenvector of the spectral decomposition of community members of the set of microbiome communities positively associated with BMI and comprising: community GMNC-2 comprising Butyricimonas, Clostridia vadinBB60 group, Oscillospirales UCG-010, Dorea, Odoribacter, Lachnospiraceae UCG-004, Oscillospiraceae UCG-002, Oscillospiraceae NK4A214 group, Christensenellaceae R-7 group, Christensenellaceae Family, Alistipes, Defluviitaleaceae UCG-011, Oscillospiraceae UCG-005, Ruminococcaceae Family, wherein the second subset of communities comprises a set of keystone taxa comprising Sutterella, Butyricimonas, Clostridia vadinBB60 group, Oscillospirales UCG-010, Dorea, Odoribacter, and with its first eigenvector of the spectral decomposition of the community members among the samples studied being positively associated with BMI.

Summary: Model-returned outputs link decreasing BMI with an increase in alpha and its association with beta diversity. Interestingly, model-returned outputs indicated that the change in beta diversity is ~6x more strongly associated with BMI or age than with medication intake, alcohol consumption or the time point studied (T1 vs T2). This suggests that, among the variables tested, BMI is the main intra-individual factor driving the change in beta diversity between T1 and T2. Similarly, the analysis of alpha diversity points towards BMI, as the main factor influencing gut microbiome diversity during the time period studied.

Through regression analyses provided by model architecture, the trained model identified associations between the abundance of 62 genera and the predicted abundances of 21 functional pathways and BMI, an 21 genera and 9 functional pathways indicated changes in their abundance between T1 and T2, which were used to generate personalized intervention plans. The model architecture was then structured to process the intersection between these two sets of results to identify genera and functional pathways that are physiological correlates of the BMI changes that occurred between T1 and T2. Eighteen genera were associated with both BMI and T1 vs T2 differences in abundance and several were associated with BMI or obesity. Christensenella and Oscillospiraceae were associated with BMI in humans. The embodiment of the model indicated a similar direction of association between these genera and BMI and also their abundances were positively correlated in our data and part of a gut microbiome community network associated with BMI.

Amongst the predicted bacterial pathways, the model indicated a significant association of simple sugar metabolism such as arabinose (MF0014), sucrose (MF0010) and melibiose (MF0009) degradation with higher BMI.

Model-returned results also point to a pattern of higher BMI being associated with higher abundance of genes related to energy extraction, supporting the idea that besides excessive calorie intake obesity development is related to the ability of the gut microbiome to extract energy from food.

Interestingly, among other pathways associated with BMI, model-returned outputs indicated propionate synthesis pathways (MF0095, MGB055, MF0094 and MGB054) to be negatively associated with BMI. Propionate is a type of short-chain fatty acid that is produced by certain types of bacteria in the gut. Increased gut propionate can reduce inflammation, improve insulin sensitivity and regulate appetite and body weight maintenance by promoting the secretion of Peptide YY (PYY) and Glucagon like peptide-1 (GLP-1).

Interestingly, 9 out of the 18 genera associated with BMI also changed their abundance in a pattern consistent with the beneficial effect of the intervention on the gut microbiome. In particular, their abundance is negatively associated with BMI and it increases at T2 or their abundance is positively associated with BMI and it decreases at T2. This finding highlights these genera as interesting targets for generating personalized interventions, involving modulations to the microbiome for improving weight-related health conditions and comorbidities in subjects. Additionaly, 9 genera that were not modulated by some aspects of a personalized intervention plan were used to identify food components, pre- and probiotics that shifted their abundance to provide improved outcomes in relation to BMI and other comorbidities.

Data analysis strategies that consider each taxa individually fail to account or consider the community assembly existing on the gut microbiome. Model architecture was thus designed to carry out networks analyses to identify groups of taxa with tightly correlated abundance patterns that may reflect the underlying microbial community assembly (i.e., where nodes represent microbial taxa and edges represent their patterns of co-abundance) and may be associated with potential consequences for human health.

The interventions generated, provided, and analyzed were shown to be effective for weight loss and during the microbiome sampling period (from T1 to T2) of this study 17.5%, 31.1%, and 14.6% of individuals lost more than 3%, 5-10%, and more than 10% of body weight, respectively. This suggests that the biomarkers identified are relevant to understand obesity and to generate personalized intervention programs to reverse obesity-related health conditions. Model associations with bacterial functional pathways relied on predicting the abundance of the relevant genes and can be related to the activity at the enzymatic or molecular level.

The digital therapeutics care programs provided to subjects, informed by genetic and baseline gut microbiome and their interactions with subject lifestyle. Personalized care program regimens generated according to model outputs were effective in achieving significant reductions of body weight across large subject pools. Different components of personalized care (e.g. fiber types, probiotics, prebiotics, dietary regimens, etc.) affect the microbial taxa identified in the models and their corresponding effect on reduction of weight, in a significant manner, which were demonstrated to guide creation of personalized treatment regimens. The platform thus generates precision dietary interventions for weight loss utilizing both genomic risk and baseline microbiome data, with digitally delivered recommendations alongside health coaching to drive active engagement of subjects for improved intervention efficacy. Moreover, the exemplary platform described, and variations thereof, can be readily implemented for digital therapeutics care for other comorbidities where genetics and gut microbiome play a role in disease etiology.

### 2.6 Methods - Model Training and Refinement

In some variations, as shown in FIG. 1B, the method 100 can further include refining the multi-omic model S170, wherein refining the multi-omic model includes: collecting a set of training data streams derived from a population of subjects, the set of training data streams capturing genetic data, microbiome data, biometric data, and lifestyle data, paired with diagnostic and therapeutic information, from the population of subjects S171, applying a set of transformation operations to the set of training data streams S172, creating a training dataset derived from the set of training data streams and the set of transformation operations S173, and training the multi-omic model in one or more stages, based upon the training dataset S174.

In order to process such data, computing platforms implementing one or more portions of the method can be implemented in one or more computing systems, wherein the computing system(s) can be implemented at least in part in the cloud and/or as a machine (e.g., computing machine, server, mobile computing device, etc.) configured to receive a computer-readable medium storing computer-readable instructions.

Data/signal inputs indicated in relation to blocks S110, S120, S130, and S140 above and/or other inputs (e.g., contextual inputs, derivative inputs, combinatorial inputs, etc.) can be used for training the multi-omic model. In more detail, features may be transformed either individually or in combination before being processed by the model(s). Combinatorial features can include microbiome-derived features, genomic (e.g., SNP, allelic variations, loci of interest)-associated features, lifestyle features, demographic features, and/or other features.

Additionally or alternatively, dynamic aspects (e.g., changes over time in features, changes in frequency between instances of respective features, other temporal aspects, other frequency-related aspects, etc.) of features derived from the samples can be used to predict or otherwise anticipate health condition statuses for generation of personalized intervention plan components.

Inputs can be aggregated from populations of subjects associated with different demographic characteristics, health statuses, health conditions, lifestyles, and/or other suitable factors.

In relation to model architecture, inputs to models described above can produce outputs that are subsequently used as inputs to an overarching model (e.g., classification model having multiple layers) that returns diagnostics, characterizations of the subject (e.g., in relation to health state, disease state, etc.), personalized intervention plan aspects (e.g., recommended dietary regimen aspects, pharmacogenomics information, prebiotics, probiotics, etc.), and/or other aspects based upon processing features in stages. However, the model(s) can implement other suitable architecture having other suitable flow for processing features derived from the inputs.

Returned classification outputs of models can include returned confidence-associated parameters in such classifications. In particular, confidence-associated parameters can have a score (e.g., percentile, other score) that indicates confidence in the returned output.

Furthermore, refined versions of the model can be configured to process fewer inputs (e.g., only a subset of inputs described above) in order to return accurate outputs for generating personalized intervention plan components associated with Blocks S150 and S160 above. Furthermore, previous features derived from inputs (e.g., new signals/signatures, interesting signals/signatures, etc.) can be returned by computing components during model refinement.

While embodiments, variations, and examples of models (e.g., in relation to inputs, outputs, and training) are described above, models associated with the method 100 can additionally or alternatively include other machine learning architecture.

Statistical analyses and/or machine learning algorithm(s) can be characterized by a learning style including any one or more of: supervised learning (e.g., using back propagation neural networks), unsupervised learning (e.g., K-means clustering), semi-supervised learning, reinforcement learning (e.g., using a Q-learning algorithm, using temporal difference learning, etc.), and any other suitable learning style.

Furthermore, any algorithm(s) can implement any one or more of: a regression algorithm, an instance-based method (e.g., k-nearest neighbor, learning vector quantization, self-organizing map, etc.), a regularization method, a decision tree learning method (e.g., classification and regression tree, chi-squared approach, random forest approach, multivariate adaptive approach, gradient boosting machine approach, etc.), a Bayesian method (e.g., naive Bayes, Bayesian belief network, etc.), a kernel method (e.g., a support vector machine, a linear discriminant analysis, etc.), a clustering method (e.g., k-means clustering), an associated rule learning algorithm (e.g., an Apriori algorithm), an artificial neural network model (e.g., a back-propagation method, a Hopfield network method, a learning vector quantization method, etc.), a deep learning algorithm (e.g., a Boltzmann machine, a convolution network method, a stacked auto-encoder method, etc.), a dimensionality reduction method (e.g., principal component analysis, partial least squares regression, etc.), an ensemble method (e.g., boosting, boot strapped aggregation, gradient boosting machine approach, etc.), and any suitable form of algorithm.

In an example, model training can be based on a cohort of subjects who lose >5% body weight while receiving personalized digital care for weight loss and present concomitant reduction of cardiovascular and/or functional bowel disorder symptomatology. A diagnostic signature can be built based on predictive variables identified in linear and/or logistic regression models on the likelihood of a subject having a disorder versus not exhibiting a disorder. Example variables include: demographics (e.g., number of coaching sessions completed, number of weight entries logged, number of Bristol stool scores logged, etc.), microbiome taxa described in any sections above, microbiome functional pathways described in any sections above, genomic SNPs described in any sections above, and any other type of variable. In the same example cohort, therapeutic signatures can be identified in models for reduction in symptomatology for different conditions described. Example variables for therapeutic signatures include demographics (e.g., gender, number of food posts, etc.), genomics markers, microbiome taxa described, and any other type of variable.

The invention(s) can also include modified variations of operations for training and refinement of other model architecture configured for improving other conditions.

### 3. System

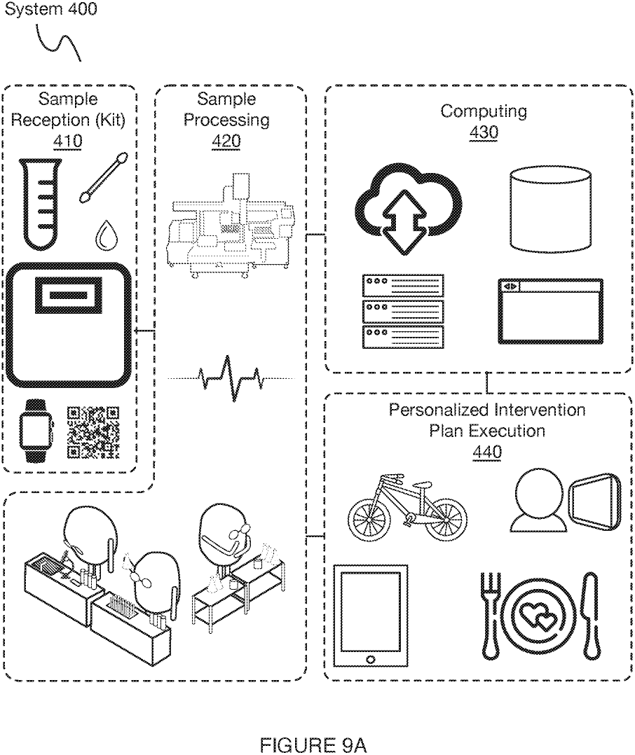

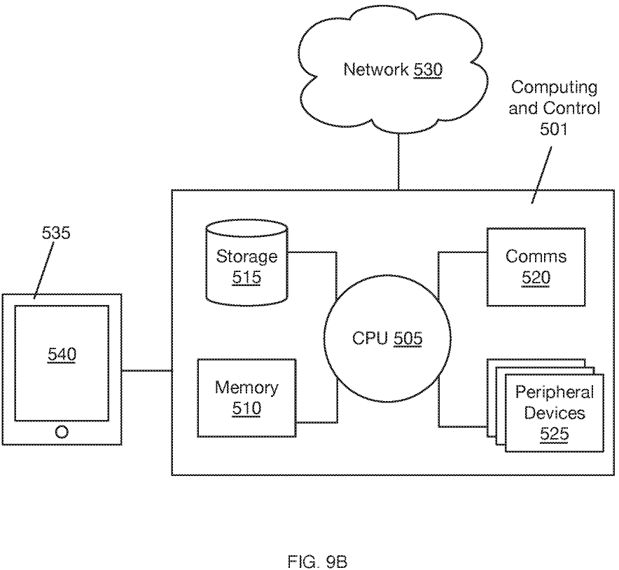

As shown in FIG. 9A, a system/platform 400 for multi-omics interventions as diagnostics, for generation of personalized therapeutic and diagnostic approaches, includes: one or more sample reception subsystems 410; one or more sample processing subsystems 420 in communication with the sample reception subsystems 410; a computing platform 430 comprising one or more processing subsystems comprising non-transitory computer-readable medium comprising instructions stored thereon, that when executed by the processing subsystems perform one or more steps of methods described above; and one or more execution subsystems 440 configured to execute components of personalized intervention plans informed by processes of the computing platform 230. In variations, the execution subsystems 440 can be configured to execute control instructions generated by the computing platform 430, where control instructions can involve instructions for controlling operation modes of one or more of: application interfaces (e.g., mobile application interfaces, web application interfaces, etc.) for signal reception, data aggregation, and/or retrieval of other inputs to be processed by system architecture; sample processing architecture (e.g., by automated/robotic subsystems), communication interfaces for performing telehealth operations, communication interfaces for providing group therapies, communication interfaces for providing counseling (e.g., through human entities, through digital entities), interfaces for providing rewards and/or other incentives to subjects, interfaces for providing and monitoring tasks provided to subjects, interfaces for connecting devices (e.g., biometric monitoring devices) of the subject with an account of the subject within the system/platform 100; interfaces for providing medications to the subject; interfaces for providing dietary advice/tracking diet behavior of the subject; and/or other suitable functionality for delivering components of a personalized intervention plan.

Embodiments of the system 400 are configured to perform one or more portions of methods described above; however, variations of the system 400 can be configured to perform other suitable methods.

The sample reception subsystem(s) 410 can include automated platforms for receiving and storing laboratory samples from sample acquisition devices of the sampling kits described above. The sample processing subsystem(s) 420 in communication with the sample reception subsystems 410 can include automated platforms for executing processing operations described above (e.g., in relation to sequencing and detection of genomic regions of interest in samples, in relation to sequencing and detection of microbiome taxa and functions of interest from samples, etc.). Such sample reception subsystems 410 and sample processing subsystems 420 can include automated and/or robotic apparatuses for transferring sample material and/or combining sample material with processing reagents, delivering processed samples to sequencing equipment, returning results from sequencing equipment for analysis by the computing platform 430, and/or other performing other suitable operations.

The computing platform 430 comprises one or more processing subsystems comprising non-transitory computer-readable medium comprising instructions stored thereon, that when executed by the processing subsystems perform one or more steps of methods described above; and one or more execution subsystems 440 configured to execute components of personalized intervention plans informed by processes of the computing platform 430.

The execution subsystems 440 can be configured to execute control instructions generated by the computing platform 430, where control instructions can involve instructions for controlling operation modes of one or more of: application interfaces (e.g., mobile application interfaces, web application interfaces, etc.) for signal reception, data aggregation, and/or retrieval of other inputs to be processed by system architecture; sample processing architecture (e.g., by automated/robotic subsystems), communication interfaces for performing telehealth operations, communication interfaces for providing group therapies, communication interfaces for providing counseling (e.g., through human entities, through digital entities), interfaces for providing rewards and/or other incentives to subjects, interfaces for providing and monitoring tasks provided to subjects, interfaces for connecting devices (e.g., biometric monitoring devices) of the subject with an account of the subject within the system/platform 400; interfaces for providing medications to the subject; interfaces for providing dietary advice/tracking diet behavior of the subject; and/or other suitable functionality for delivering components of a personalized intervention plan.

In particular, the execution subsystems 440 are structured to implement a next-generation, prescription-grade, digital therapeutics program that uses artificial intelligence (AI) to analyze genetics, gut bacteria, lifestyle habits, socioeconomic and behavioral risk patterns to create evidence-based personalized nutrition, fitness, sleep and stress management program to reduce weight, reverse weight-related illnesses, and improve mental health. In examples, the execution subsystems 440 are structured to execute digital precision care interventions by mobile applications of subject devices. The execution subsystems 440 include non-transitory media storing computer-readable instructions and architecture for a genomic loci and gut microbiome-informed health program that is geared primarily toward individuals who suffer from health conditions described (e.g., mental health conditions, weight-related condition).

FIG. 9B depicts an embodiment of a computing and control system 501 configured to execute one or more portions of methods described. The computing and control subsystem 501 can be programmed or otherwise configured to, for example, extract, receive, and process input data with model architecture, and to return components of a personalized care program for a subject that can be delivered (e.g., as a prescription digital therapeutic).

The computing and control subsystem 501 includes architecture for regulating various aspects of sample/data processing, personalized care program generation, and other functionalities of the present disclosure. The computing and control subsystem 501 can be an electronic device of a user or a computer system that is remotely located with respect to the electronic device. The electronic device can be a mobile electronic device.

The computing and control subsystem 501 includes a central processing unit (CPU, also “processor” and “computer processor” herein) 505, which can be a single core or multi core processor, or a plurality of processors for parallel processing. The computing and control subsystem 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet.

In some embodiments, the network 530 is a telecommunication and/or data network. The network 530 can include one or more computer servers, which can enable distributed computing, such as cloud computing. For example, one or more computer servers may enable cloud computing over the network 530 (“the cloud”) to perform various aspects of facilitating charging of an electric vehicle, with desired security, authentication, and locking functionalities associated with various types of charging sessions and/or different users. Such cloud computing may be provided by cloud computing platforms such as, for example, Amazon Web Services (AWS), Microsoft Azure, Google Cloud Platform, and IBM cloud. In some embodiments, the network 530, with the aid of the computer system 501, can implement a peer-to-peer network, which may enable devices coupled to the computer system 501 to behave as a client or a server.

The CPU 505 can include one or more computer processors and/or one or more graphics processing units (GPUs). The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback. The CPU 505 can be part of a circuit, such as an integrated circuit. One or more other components of the computing and control subsystem 501 can be included in the circuit. In some embodiments, the circuit is an application specific integrated circuit (ASIC).

The storage unit 515 can store files, such as drivers, libraries and saved programs. The storage unit 515 can store user data, e.g., user preferences and user programs. In some embodiments, the computer system 501 can include one or more additional data storage units that are external to the computer system 501, such as located on a remote server that is in communication with the computer system 501 through an intranet or the Internet.

The computing and control subsystem 501 can communicate with one or more remote computer systems through the network 530. For instance, the computer system 501 can communicate with a remote computer system of a user. Examples of remote computer systems include personal computers (e.g., portable PC), slate or tablet PC’s (e.g., Apple® iPad, Samsung® Galaxy Tab), telephones, Smart phones (e.g., Apple® iPhone, Android-enabled device, Blackberry®), or personal digital assistants. The user can access the computer system 501 via the network 530.

Methods as described herein can be implemented by way of machine (e.g., computer processor) executable code stored on an electronic storage location of the computing and control subsystem 501, such as, for example, on the memory 510 or electronic storage unit 515. The machine executable or machine-readable code can be provided in the form of software. During use, the code can be executed by the processor 505. In some embodiments, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Embodiments of the systems and methods provided herein, such as the computing and control subsystem 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, or disk drives, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as the main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computing and control subsystem 501 can include or be in communication with an electronic display 535 that comprises a user interface (UI) 540 for providing, for example, a visual display indicative of statuses associated with charging of an electric vehicle, security information, authentication information, and locking statuses associated with various types of charging sessions and/or different users. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 505. The algorithm can, for example, facilitate charging of an electric vehicle, with desired security, authentication, and locking functionalities associated with various types of charging sessions and/or different users.

In one set of embodiments, methods implemented by way of or as supported by the computing and control subsystem 501 can include methods for sample/data processing, personalized care program generation, and other functionalities. Communicated statuses can then be used by the system 100 to return notifications and/or execute other actions for providing personalized care.

Additionally or alternatively, the computing and control subsystem 501 can include architecture with programming to execute other suitable methods.

### 4. Conclusions

Embodiments of the invention(s) can include every combination and permutation of the various system components and the various method processes, including any variants (e.g., embodiments, variations, examples, specific examples, figures, etc.), where portions of embodiments of the methods and/or processes described herein can be performed asynchronously (e.g., sequentially), concurrently (e.g., in parallel), or in any other suitable order by and/or using one or more instances, elements, components of, and/or other aspects of the system and/or other entities described herein.

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments of the methods and/or systems can be embodied and/or implemented at least in part as a machine configured to receive a computer-readable medium storing computer-readable instructions. The instructions can be executed by computer-executable components that can be integrated with embodiments of the system. The computer-readable medium can be stored on any suitable computer-readable media such as RAMs, ROMs, flash memory, EEPROMs, optical devices (CD or DVD), hard drives, floppy drives, systems for cloud computing, or any suitable device. The computer-executable component can be a general or application specific processor, but any suitable dedicated hardware or hardware/firmware combination device can alternatively or additionally execute the instructions.

As a person skilled in the art will recognize from the previous detailed description and from the figures and claims, modifications and changes can be made to embodiments of the methods, systems, and/or variants without departing from the scope defined in the claims. Variants described herein are not meant to be restrictive. Certain features included in the drawings may be exaggerated in size, and other features may be omitted for clarity and should not be restrictive. The figures are not necessarily to scale. The absolute or relative dimensions or proportions may vary. Section titles herein are used for organizational convenience and are not meant to be restrictive. The description of any variant is not necessarily limited to any section of this specification.

What is claimed is:

1. A method for prevention and treatment of a mental health condition, the method comprising:
simultaneously reducing severity of a set of mental health condition symptoms by at least 50% and producing a reduction in body mass index (BMI) greater than 2 BMI units, across a set of subjects upon:
receiving a set of samples from the set of subjects;
receiving a biometric dataset from the set of subjects;
receiving a lifestyle dataset from the set of subjects;
returning a genomic single nucleotide polymorphism (SNP) profile, a baseline microbiome state, and a set of signatures upon processing the set of samples, the biometric dataset, and the lifestyle dataset with a set of transformation operations;
generating a personalized intervention plan for a subject of the set of subjects upon processing the genomic SNP profile, the baseline microbiome state, and the set of signatures with a multi-omic model; and
executing the personalized intervention plan for the subject.

2. The method of claim 1, wherein the set of mental health condition symptoms comprises symptoms of anxiety, depression, and sleep.

3. The method of claim 2, wherein the method comprises simultaneously reducing severity of anxiety symptoms by at least 20%, depression symptoms by at least 20%, and insomnia symptoms by at least 20% across the population of subjects.

4. The method of claim 1, wherein the set of samples comprises a saliva sample and a gut sample, and wherein the set of transformation operations comprises nucleic acid extraction, purification, and genotyping for the saliva sample, and amplification of and generating a set of sequencing reads of 16S rRNA V3-V4 regions for the gut sample.

5. The method of claim 4, wherein the set of transformation operations further comprises demultiplexing the set of sequencing reads, generating amplicon sequence variants (ASVs) from data derived from the set of sequencing reads, performing taxonomic and functional annotation of data derived from the set of sequencing reads based on alignment methods and graph-based methods, performing linear and nonlinear dimensionality reductions with data derived from the set of sequencing reads, and performing at least one of machine learning and statistical inference methods upon data derived from the set of sequencing reads to derive informative features from the baseline microbiome state.

6. The method of claim 4, wherein the set of transformation operations further comprises modeling microbiome data derived from the set of samples as a network, and organizing microbial communities, from the network, into clusters of at least one of amplicon sequence variants, taxa, and pathways, wherein the clusters have intracluster correlations greater than intercluster correlations.

7. The method of claim 1, further comprising: returning a set of SNP features and a set of microbiome features for each of the set of subjects from the multi-omic model, and generating the personalized intervention plan from the set of SNP features and the set of microbiome features.

8. The method of claim 7, wherein the set of SNP features comprises characterizations of risk alleles detected for the subject, said risk alleles individually or in combination used to determine inherited risk for at least one of: irritable bowel syndrome, obstructive sleep apnea, alcohol use disorder, major depressive disorder, and type I diabetes.

9. The method of claim 8, wherein the set of SNP features used to determine inherited risks comprises risk alleles associated with Genome Wide Association Studies (GWAS) corresponding to: risk allele variants for irritable bowel syndrome from GWAS study GCST90016564, risk allele variants for obstructive sleep apnea from GWAS study GCST011921, risk allele variants for alcohol use disorder from GWAS study GCST012354, risk allele variants for major depressive disorder from GWAS study GCST007342, and risk allele variants for type I diabetes from GWAS study GCST90013445.

10. The method of claim 7, wherein the set of microbiome features comprises features associated with Dorea genus, Ruminococcaceae genus UBA1819, Oscillospiraceae genus UCG003, Eubacterium ventriosum group, Ruminococcaceae genus DTU089, Prevotella, and Adlercreutzia in relation to anxiety symptoms of the set of mental health condition symptoms; features associated with Clostridium innocuum group, Oscillospiraceae genus UCG003, Anaerostipes, Eubacterium ventriosum group, Lactobacillus, Negativibacillus, Prevotella, Oscillibacter, and Actinomyces in relation to depression symptoms of the set of mental health condition symptoms; and features associated with Butyricimonas and Roseburia in relation to sleep symptoms of the set of mental health condition symptoms.

11. The method of claim 1, wherein receiving the biometric dataset comprises:
receiving a bodyweight value from at least one of the population of subjects, generated from a digital weighing scale, wherein the biometric dataset comprises a BMI value, and
receiving a blood glucose value from at least one of the population of subjects, generated from a continuous glucose monitor, wherein the biometric dataset comprises a blood glucose value.

12. The method of claim 1, wherein the personalized intervention plan is delivered digitally through a mobile device application.

13. The method of claim 12, wherein the personalized intervention plan comprises a dietary plan configured to adjust taxonomic abundances and microbiome function represented by microorganism taxa of the baseline microbiome state, wherein the dietary plan comprises a recommended food list comprising superfood items, items to consume in moderation, items to avoid consuming, items associated with conditions comorbid with a set of mental health condition symptoms, and items associated with insulin resistance.

14. The method of claim 1, wherein the personalized intervention plan comprises provision of a prebiotic blend including ingredients rich in at least one of: inulin, flavonoids, withanolides, sitoindosides or acylsterylglucosides according to a regimen of consumption.

15. The method of claim 1, wherein, based on detection of gene CYP2C19 from the genomic SNP profile, the personalized intervention plan provides pharmacogenomics information for Citalopram, Escitalopram, Sertraline, Amitriptyline, Clomipramine, Doxepin, Imipramine, and Trimipramine.

16. The method of claim 1, wherein, based on detection of gene CYP2B6 from the genomic SNP profile, the personalized intervention plan provides pharmacogenomics information for Bupropion.

17. The method of claim 1, wherein based on detection of gene CYP2C9 from the genomic SNP profile, the personalized intervention plan provides pharmacogenomics information for Phenytoin.

18. The method of claim 1, wherein, for the reduction in BMI, the personalized intervention plan is generated based upon detection of a set of microbiome taxa from the baseline microbiome state, associated with a) a first subset of taxa that have a negative association with BMI and comprising: Solobacterium, Eubacterium ruminantium group, Erysipelatoclostridiaceae UCG 004, Catenibacterium, Desulfovibrio, Enterobacter, Eubacterium nodatum group, Paraprevotella, Desulfovibrionaceae Family, Hydrogenoanaerobacterium, Coriobacteriales Incertae Sedis Family, Oscillospiraceae UCG 002, Eubacterium siraeum group, Christensenellaceae R 7 group, Phascolarctobacterium, Clostridia vadinBB60 group, Ruminococcaceae Family, Barnesiella, Oscillospiraceae UCG 005, Caproiciproducens, Ruminococcaceae UBA1819, Ruminiclostridium, Erysipelatoclostridium, Oscillospiraceae Family, Christensenellaceae Family, Candidatus Soleaferrea, Alistipes, Marvinbryantia, Lachnospiraceae NK4A136 group, Oscillospiraceae NK4A214 group Anaerovoracaceae Family XIII AD3011 group, Eggerthella, Anaerofilum, Gordonibacter, and b) a second subset of taxa that have a positive association with BMI and comprising: Actinomyces, Lachnoclostridium, Granulicatella, Fusicatenibacter, Lachnospiraceae Family, Streptococcus, Lachnospiraceae CAG 56, Lachnospiraceae UCG 001, Sutterella, Bifidobacterium, Oscillospiraceae UCG 003, Dialister, Prevotella, Eggerthellaceae Family, Candidatus Stoquefichus, Sellimonas, Ruminococcus gauvreauii group, Roseburia, Oscillospira, Enterorhabdus, Muribaculaceae, Allisonella, Agathobacter, Peptococcus, Acidaminococcus, Fenollaria, Megasphaera, and Cloacibacillus.

19. The method of claim 1, wherein, for the reduction in BMI, the personalized intervention plan is generated based upon detection of microbial genes associated with a) a first subset of pathways that have a negative association with BMI and comprising: putrescine degradation, GABA synthesis, phenylalanine degradation, Histamine synthesis, triacylglycerol degradation, lysine degradation, Propionate synthesis, lactate consumption, mucin degradation and serine degradation; and b) a second subset of pathways that have a positive association with BMI and comprising: sucrose degradation, fructan degradation, melibiose degradation, arabinose degradation, hydrogen metabolism, arabinoxylan degradation, simple sugar metabolism, and cysteine biosynthesis/homocysteine degradation.

20. The method of claim 1, wherein, for the reduction in BMI, the personalized intervention plan is generated based upon detection of microbial genes associated with a set of microbiome communities, comprising a) a first subset of communities having a first eigenvector of a spectral decomposition of community members of the set of microbiome communities negatively associated with BMI and comprising: community GMNC-3; and b) a second subset of communities having the first eigenvector of the spectral decomposition of community members of the set of microbiome communities positively associated with BMI and comprising: community GMNC-2 comprising Butyricimonas, Clostridia vadinBB60 group, Oscillospirales UCG-010, Dorea, Odoribacter, Lachnospiraceae UCG-004, Oscillospiraceae UCG-002, Oscillospiraceae NK4A214 group, Christensenellaceae R-7 group, Christensenellaceae Family, Alistipes, Defluviitaleaceae UCG-011, Oscillospiraceae UCG-005, Ruminococcaceae Family, wherein the second subset of communities comprises a set of keystone taxa comprising Sutterella, Butyricimonas, Clostridia vadinBB60 group, Oscillospirales UCG-010, Dorea, Odoribacter, and with its first eigenvector of the spectral decomposition of the community members among the samples studied being positively associated with BMI.

21. The method of claim 1, further comprising determining a response to the personalized intervention plan by the subject, based upon identification of modulation of microbial taxa associated with a combination of a) a first subset of taxa that decrease in abundance longitudinally in time and comprising: Parabacteroides, Desulfovibrio, Oscillospiraceae UCG-002, Sutterella, Akkermansia, DTU014, Anaerotruncus, Unannotated Erysipelotrichaceae Family, Clostridia vadinBB60 group, Eubacterium fissicatena group, Enterorhabdus, Christensenellaceae R 7 group, Clostridium sensu stricto 1, Oscillospirales UCG-010, Megasphaera, Unannotated Rhodospirillales Order, Unannotated Erysipelatoclostridiaceae Family, Coprobacter, Rothia, Cloacibacillus, Enterobacter that increase longitudinally, and Peptostreptococcus, Fenollaria, Paludicola, Holdemanella, Erysipelatoclostridiaceae UCG004, Agathobacter, Eubacterium ruminantium group, Enterococcus, RF39, Anaerofustis, Lachnoclostridium, Eggerthella, Phascolarctobacterium, Roseburia, Solobacterium; b) a second subset of taxa that decrease in abundance longitudinally in time and comprising gut microbial communities associated with GMNC-1, GMNC-2 and GMNC-4; and a third subset of taxa that increase in abundance longitudinally in time and comprising gut microbial communities associated with GMNC-3.

22. The method of claim 1, wherein, for the reduction in BMI, the efficacy of the personalized intervention plan is determined based on modulation of microbial genes associated with Histamine Synthesis, Nitric oxide degradation in relation to nitric oxide reductase and nitric oxide dioxygenase, Inositol degradation, 4-aminobutyrate degradation, pyruvate dehydrogenase complex, Nitric oxide synthesis in relation to nitrite reductase, and GABA degradation.

23. The method of claim 1, wherein the set of transformation operations comprises generating a mental health composite score derived from a weighted aggregation of a set of features comprising: a Shannon Diversity Index, an Acetate biosynthesis feature represented in the set of samples, a Butyrate biosynthesis feature represented in the set of samples, a Propionate biosynthesis feature represented in the set of samples, a GABA biosynthesis feature represented in the set of samples, and a Tryptophan synthesis feature represented in the set of samples.

24. A method for prevention and treatment of a mental health condition, the method comprising:
simultaneously reducing severity of a set of mental health condition symptoms, comprising symptoms of anxiety, depression, and sleep, by at least 50% across a population of subjects upon:
receiving a set of samples from the population of subjects, the set of samples comprising saliva samples and gut samples;
receiving a biometric dataset from the population of subjects, the biometric dataset comprising BMI values;
receiving a lifestyle dataset from the population of subjects;
returning a genomic single nucleotide polymorphism (SNP) profile, a microbiome state, and a set of signatures upon processing the set of samples, the biometric dataset, and the lifestyle dataset with a set of transformation operations;
generating a personalized intervention plan for a subject of the set of subjects upon processing the genomic SNP profile, the microbiome state, and the set of signatures with a multi-omic model; and
executing the personalized intervention plan for the subject.

25. A method for prevention and treatment of a health condition, the method comprising:
producing a reduction in body mass index (BMI) greater than an average 2 BMI units across a population of subjects upon:
receiving a set of samples from the population of subjects, the set of samples comprising saliva samples and gut samples;
receiving a biometric dataset from the population of subjects, the biometric dataset comprising BMI values and blood glucose values;
receiving a lifestyle dataset from the population of subjects;
returning a genomic single nucleotide polymorphism (SNP) profile, a microbiome state, and a set of signatures upon processing the set of samples, the biometric dataset, and the lifestyle dataset with a set of transformation operations;
generating a personalized intervention plan for a subject of the set of subjects upon processing the genomic SNP profile, the microbiome state, and the set of signatures with a multi-omic model; and
executing the personalized intervention plan for the subject.
